(19) United States
(12) Patent Application Publication
(10) Pub. No.: US 2008/0160496 A1
(43) Pub. Date: Jul. 3, 2008

(54) Preserved Viable Cartilage, Method for Its Preservation, and System and Devices Used Therefor

(76) Inventors: Victor Rzepakovsky, Ness Zionna (IL); Ginadi Shaham, Yavneh (IL); Udi Damari, Ganiey Tikva (IL); Sachi Norman, Gani Tikva (IL); Peter Bartal, Yavne (IL)

Correspondence Address: NATH & ASSOCIATES, 112 South West Street, Alexandria, VA, 22314, US

(21) Appl. No.: 11/884,767

(22) PCT Filed: Feb. 22, 2006
(86) PCT No.: PCT/IL2006/000231

§ 371 Date: Dec. 31, 2007

Related U.S. Application Data
(60) Provisional application No. 60/654,454, filed on Feb. 22, 2005.

(51) Int. Cl. A01N 1/02; A61F 2/02; A61B 17/32; A61B 19/00

(52) U.S. Cl. 435/1.3; 623/23.72; 606/167; 206/438

(57) ABSTRACT

The present invention provides methods for providing cartilage-containing tissue for grafting, comprising providing excised cartilage-containing tissue; and treating said excised cartilage-containing and cryogenically preserving the treated cartilage-containing tissue under appropriate cryogenic preservation conditions so as to yield cryogenically preserved cartilage-containing tissue having at least 10% viable chondrocytes throughout the cartilage portion of the cartilage-containing tissue after preservation, as tested in a live/dead ratio assay. Treatment may comprise providing one or a plurality of incisions in said cartilage portion to a predetermined depth therein and/or introducing a cryoprotectant agent at least into said cartilage portion. The invention also provides viable cartilage obtainable by the methods of the invention, methods of grafting such preserved, viable cartilage containing tissue in a recipient, as well as apparatuses, vessels and systems for preparing a cartilage-containing tissue for cryogenic preservation and subsequent grafting in a recipient.

### FIELD OF THE INVENTION

This invention relates to the cryogenic preservation of cartilage-containing tissue, including human tissue.

### LIST OF REFERENCES

The following references are brought to facilitate description of the background of the present invention, and should not be construed as limiting the patentability of the invention:
McGoveran, B. M. et al., The Journal of Knee Surgery, vol. 15, No. 2 Spring 2002;
Muldrew, K. et al., Cryobiology 43, 260-267 (2001);
Muldrew, K. et al., Cryobiology 31, 31-38 (1994);
Muldrew et al. Cryobiology 40, 102-109 (2000)
Williams, S K. et al, The Journal of Bone and Joint Surgery (American), 85:2111-2120 (2003).
U.S. Pat. No. 5,131,850 to Kelvin G. M.;
U.S. Pat. No. 6,740,484 to Khirabadi et al.
PCT Application No. IL2004/000929 to Damari et al.
Glaser C. and Putz R., Osteoarthritis and Cartilage 10: 83-99 (2002)
Williams S., (2004), American Academy of Orthopedic Surgeons Poster Presentations

### BACKGROUND OF THE INVENTION

Adult cartilage is a connective tissue populated by chondrocytes embedded in a dense extra cellular matrix (ECM) composed of a collagenous fiber network. The ground substance of cartilage is rich in proteoglycan molecules consisting of a core protein with numerous (about 100) glycosaminoglycans (GAGs) attached in a bottle-brush fashion around it. GAGs are made of repeating units of disaccharides, one of which is always a glycosamine (hence the name) such as glucosamine or galactosamine. In cartilage, the GAGs attached to the core proteins are mainly chondroitin sulfate (CS) and keratan sulfate (KS).

Between 60 and 80 percent of the net weight of cartilage is water, and this large component of water accounts for the elastic nature of cartilage. Water is attracted to the negative charges in the abundant sulfate and carboxyl groups on the GAGs. This hydration permits diffusion of water-soluble molecules in the ground substance. However the movement of large molecules and bacteria is inhibited. Cartilage is poorly vascularized, and gets most of its nutrients through diffusion. It is noted that this high content of water is one of the important factors that hinder successful cryopreservation, as discuss below.

It has been shown that mechanical pressure can remove water from cartilage. Application of pressure was found to lead to compression of the proteoglycan-water pads consistent with fluid flow away from the loaded area (Glaser and Putz, 2002).

Functional articular cartilage is critical to proper joint function. Unfortunately, articular cartilage has low self-repair ability and therefore defects are prone to cause abnormal joint biomechanics, leading in the long run to degenerative changes. Damage to cartilage is partially healed by the bone at the bone-cartilage junction, where fibro-cartilage is produced. Thus, where the damaged area is relatively small, a surgeon may remove the damaged cartilage and cause intentional minor damage to the bone in order to accelerate natural healing. However, the tissue produced by the bone is normally a relatively rigid scar, and this process is not applicable to larger lesions.

One method to repair cartilage damage is the implantation of osteochondral tissue to replace the damaged tissue. Normally, the implanted tissue (comprising bone and cartilage) is taken from a cadaver, from a site that is most similar to the organ that is in need of repair in the recipient. This allows the implanted tissue to have the most similar shape, arrangement (e.g. of bone and cartilage tissue) and weight bearing characteristics as the tissue of the implant site. This implant (or graft) is often named “allograft” since the graft is taken from one individual and implanted in another.

Post-implantation viability of the chondrocytes is necessary for the long-term maintenance of the biomechanical properties of the cartilage graft. Chondrocytes in cartilage are enclosed in lacunas within the extra-cellular matrix (ECM), such that if a cell dies within a lacuna it cannot be replaced by a cell migrating thereto. Thus, unlike bone grafts that may comprise dead bone tissue (which will be later populated by bone cells that would migrate into the implant) the cartilage graft must provide viable cartilage cells embedded in the cartilage ECM. Viability of cartilage cells is reduced from the moment of harvesting, therefore it is best to transplant a cartilage-containing tissue immediately following harvesting. Although tissue banks sometimes provide cartilage grafts which are up to 45 days after harvesting (usually with very low if any viable cells), it is commonly accepted that cartilage cells can be maintained viable within cartilage for a restricted period of time and should therefore be transplanted within a few days (no more than 14) from the moment of harvesting (Williams et al., 2003; U.S. Pat. No. 5,131,850). The restricted storage period does not normally allow sufficient time to test the donated tissue for undesired agents or traits such as transmittable diseases. It also reduces the chances of finding the best donor-recipient match (in terms of graft condition and shape as well as graft rejection).

Currently, transplantation of viable cartilage is limited to grafts that were preserved for relatively a short period, and were maintained at a temperature above freezing. Long term banking, by way of freezing, while maintaining viability of the graft has not been described to date.

Articular cartilage is structurally divided to three layers: a superficial layer being the outermost portion (furthest from the bone) an intermediate layer and a deep layer of the cartilage that is adjacent to the bone. The cells in each layer have different shapes, and the chondrocytes in the intermediate layer are distinguished from those in the deep or superficial layers by being more susceptible to freeze-thaw injury (Muldrew et al., 1994).

Muldrew et al., (Muldrew et al., 2000) showed that the layer of about 40 μm from the surface allows non-planar ice crystals to be formed thus allowing recovery of cells that are less than about 50 μm away from any surface. In this work Muldrew et al. disclosed that cutting the cartilage portion of a cartilage-containing bone plug lead to survival of cells that were up to 50 μm away from the cut surface, but concluded that such cut cartilage would not be suitable for grafting.

In a later work done by Muldrew et al. (2001) cartilage was cryopreserved by exposure of the cartilage to step-wise decreasing external temperature. This method resulted in improved chondrocytes recovery within the thawed cartilage to a maximum depth of 200 μm from the surface of the cartilage. However, cartilage that was further from the cartilage surface did not survive, and the overall survival of chondrocytes was less than 20% (since cells survived only in up to 200 μm from the surface, and sheep cartilage normally measures about 1 mm). In addition, there was considerable variability in the cells' survival rates within the experimental group and the mean cell recovery was not appreciably improved.

U.S. Pat. No. 6,740,484 disclosed a method for vitrifying tissue (including cartilage segments). Vitrification means solidification, as in glass, without ice crystal formation. This was done by raising the glass transition temperature and reducing homogenous nucleation temperature, by adding cryoprotectants at high concentrations. This publication further disclosed high survival rates for chondrocytes embedded in cartilage ECM, as determined by the Alamar Blue method.

Finally, in WO2005/032251 the “Multi-temperature gradient” (MTG) directional solidification (or directional freezing) was employed to freeze osteochondral cartilage-containing bone plugs taken from sheep. The number of live cells observed in thawed cartilage was up to almost 70% of the number observed in the fresh sample (tested by the live/dead ratio assay). The cartilage thus frozen was viable and was successfully grafted in sheep. This work was done with sheep articular cartilage that is normally about 1 mm thick.

### SUMMARY OF THE INVENTION

Some terms used herein and their meanings are as follows:

The term “cartilage-containing tissue” in the context of this invention means any tissue, natural or synthetic, comprising at least viable cartilage cells (chondrocytes), and thus also includes cartilage tissue. According to one embodiment, the cartilage cells are embedded in cartilage extra cellular matrix (ECM), whether or not comprising other natural, artificial or bio-artificial elements including cells of other types and/or ECM. Such cartilage-containing tissue may be taken from any source, including, for example, hyaline cartilage (such as the articular cartilage present in the tip of joints, such as hip, knee, shoulder, elbow, etc.) and fibrocartilage (such as the cartilage present in the ears and in the inner parts of the nose). It may be for example menisci or an osteochondral tissue (i.e. tissue comprising both cartilage and bone). Osteochondral tissue is often harvested or grafted in the form of an osteochondral plug or osteochondral cylinder. However, cartilage-containing tissue of the present invention may also include lager structures, for example the whole condyle, namely the rounded protrusion at the end of a bone, sometimes referred to as a hemi-condyle. Non limiting examples for non-cartilage cells and tissues that may be included in a viable cartilage sample are cells and/or extra cellular matrix of bone, tendon, ligament, etc.

The term “excised cartilage-containing tissue” means cartilage-containing tissue that was removed from a live or dead donor.

The terms “viable cells” and “viable tissue” in the context of this invention mean (as the context requires) cells or tissue comprising cells that are capable of surviving and maintaining their original function provided that they are given the necessary conditions (e.g. nutrients, temperature and the like). When applied to frozen cells/tissue, the term “viable” denotes such cells or tissues that are capable of remaining viable after being thawed. In the present invention, viability of cells is determined by a live/dead ratio assay as described below. When measured in vivo, meaning after transplantation, the determination may also include assays that are known in the art and to give evidence to the functionality of the chondrocytes; such evidence can be maintenance of the structure of ECM, production of hyaline matrix (which can be produced only by chondrocytes) etc. In order for the cartilage containing tissue to be deemed viable, at least some of the chondrocytes embedded therein must be viable, preferably 10% or more, 20% or more, 30% or more, 40% or more, 50% or more, 65% or more or even 69% or more (e.g. using the live/dead ratio assay detailed below). When viability is said to be “throughout the cartilage portion of the cartilage-containing tissue” it is meant that viable cells are found essentially in all the area of the cartilage portion of the cartilage-containing tissue (i.e. not localized at one part such as the top of the cartilage), and the percentage is the average viability of the whole cartilage portion. When the cartilage-containing tissue is osteochondral tissue (e.g. plug, condyle or hemi-condyle), this also means that viable chondrocytes are found in all three layers of the cartilage and the specified percentage of viability is applicable to each and every layer.

The term “surface of the cartilage portion” means any edge, surface or any other part of the cartilage portion, exposed or not, including the parts of the cartilage that were previously joined to the donor and which represent where it was cut from a donor, the surface of the incisions that may be introduced within the cartilage-containing tissue as disclosed in the present invention, and in cases where the cartilage-containing tissue comprises bone or other natural or artificial structures, also the boundary between the cartilage portion and the bone or other natural or artificial structure.

The term “cryogenic preservation” denotes a process including at least one step of lowering the temperature of cartilage-containing tissue from a temperature that is above the freezing temperature of the biological material (or the solution in which it is immersed) to a temperature that is below that freezing temperature. Cryopreservation encompasses freezing and vitrification. In order to reduce the damage to cells or tissue during cryogenic preservation, at least one (intracellular and/or extra-cellular) cryoprotectant agent (CPA) is normally added to the tissue before preservation. These are substances that increase the cells' ability to withstand the cryogenic preservation, storage and/or thawing, by any manner including by stabilization of cell membranes or replacement of the water content of the cells. Non-limiting examples of CPAs include glycerol, DMSO, Ethylene Glycol, Propylene Glycol (1,2 Propandyol), Acetamide, Methanol, Butanediol, sugars such as dextran, glucose, fructose, sucrose, trehalose, macromolecules such as Poly Vinyl Pyrrolidone (PVP), hydroxy ethyl starch (HES), albumin, serum, antifreeze protein and antioxidants. The term “freezing” denotes a process of cryogenic preservation that causes the formation of ice crystals within the frozen material.

The term “appropriate cryogenic preservation conditions” means such conditions that would cause freezing and/or vitrification of the cartilage-containing tissue, in such manner that would maintain, after thawing, at least some of the cartilage cells in a viable state. In one aspect, “appropriate cryogenic preservation conditions” also include such conditions that would prevent the formation of planar ice in the cartilage portion of the cartilage-containing tissue to be 20% or more of the weight or the volume of the cartilage portion of the tissue. Such conditions relate to the solution in which the cartilage-containing tissue is maintained (including freezing or vitrification solutions) and its constituents, the freezing or vitrification protocol including rate of cooling, temperature regime, directional freezing, stationary freezing, controlled rate freezing or uncontrolled freezing, etc. as known in the art. Non-limiting embodiments for such appropriate conditions include those embodiments and examples described herein. Accordingly, “preserved cartilage-containing tissue” means cartilage-containing tissue that was frozen or vitrified at some point, regardless whether or not the cartilage-containing tissue was also thawed or otherwise manipulated. Such a preserved tissue can be preserved at sub-zero temperatures for a long term period which can range from one day to, theoretically, infinity.

The term “live/dead ratio assay” means an assay using dyes which differentially dye live cells and dead cell. One non limiting example for such assay is the SYTO-13/Propidium Iodide (PI) assay of Molecular probe Inc., USA, used according to the manufacturer's manual to obtain dual parameter fluorescence histograms. In this assay live cells are colored fluorescent green and dead ones—fluorescent red.

The term “cartilage portion” as used herein includes the superficial layer of the cartilage containing tissue, optionally, and at times, preferably, at least a portion of the intermediate layer of the cartilage containing tissue or even at least a portion of the deep later thereof.

The term “biomechanical properties” as used herein denotes a quantitative measure to evaluate the ability of the cartilage after implantation to withstand mechanical pressure during normal knee function. One non-limiting example for such a method includes unconfined compression testing, as known in the art.

This invention discloses methods, systems and apparatuses for cryogenically preserving cartilage that may be used for any purpose, such as for grafting or as a source for extraction of cartilage cells (chondrocytes). These methods have shown to allow long term preservation of cartilage, which in turn allows, inter alia, adequate time for testing for pathogens and donor/recipient compatibility. In addition, these methods, systems and apparatuses allow creating a bank of human cartilage-containing tissue for future transplantation needs, which allow selecting better cartilage not only in terms of donor-recipient compatibility but also as relating to the compatibility of shape between recipient and donor and the condition of the tissue (younger, intact cartilage being preferred). Such bank may also provide a source for cartilage cells that may be extracted from the banks cartilage containing tissue and used for any purpose, including to the preparation of bio-artificial chondrocyte containing tissue.

Accordingly, by a first aspect the present invention provides a method for providing viable cartilage-containing tissue, comprising:
(a) providing excised cartilage-containing tissue; and
(a) treating said excised cartilage-containing and cryogenically preserving the treated cartilage-containing tissue under appropriate cryogenic preservation conditions so as to yield cryogenically preserved cartilage-containing tissue having at least 10% viable chondrocytes throughout the cartilage portion of the cartilage-containing tissue after preservation, as determined in a live/dead ratio assay.

The viable chondrocytes that are in the cartilage-containing tissue after treatment followed by preservation may be found in every layer of the cartilage, including the intermediate and deep layer. In fact, as a result of said treatment viable chondrocytes may be 50 μm from any surface of the cartilage portion of the cartilage-containing tissue, and even 75 μm, 100 μm, 150 μm, 200 μm from the surface, or even farther from the surface and deeper from the surface of the cartilage containing tissue. These distances may be measured not only in respect of the cartilage upper surface (or the periphery of the excised cartilage-containing tissue) but also for cells embedded 200 μm or deeper in the intermediate or deep layer.

In addition to the live/dead ratio assay, biomechanical parameters of the cartilage, such as withstanding mechanical pressure can be estimated in comparison to fresh tissue using a confined compression test. According to one embodiment, the biomechanical parameter may be the maintenance of least 50% of the elastic strength of the matrix surrounding the cells.

Thus, by an alternative aspect, the present invention provides a method for providing viable cartilage-containing tissue, comprising:
(a) providing excised cartilage-containing tissue having a cartilage portion;
(b) treating said excised cartilage containing tissue by providing at least one incision in said cartilage portion to a predetermined depth therein; and
(c) cryogenically preserving the treated cartilage-containing tissue under appropriate cryogenic preservation conditions.

Thus, treatment of the cartilage containing tissue before cryopreservation may include providing at least one incision in a cartilage portion of the cartilage containing tissue to a predetermined depth therein. The predetermined depth may be 50 μm from any surface of the cartilage portion of the cartilage-containing tissue, and even 75 μm, 100 μm, 150 μm, 200 μm from the surface and deeper. It may be preferable for some embodiments, however, that the predetermined depth does not exceed the local thickness of the cartilage portion, i.e. does not penetrate the bone portion of the cartilage plug.

According to one embodiment, said treatment includes providing a plurality of incisions in the cartilage containing tissue formed by means of cutting blades applied to the cartilage containing tissue. In other embodiments the incisions may be formed using suitable lasers, pins, or any other incision-forming elements.

According to another embodiment, the plurality of incisions is provided in an incision pattern over the cartilage portion, the incision pattern comprises a plurality of individual incisions. The incision pattern may include, without being limited thereto, a plurality of substantially elongate channels in substantially parallel spaced relationship; a plurality of substantially elongate channels radiating from a common central area; a plurality of substantially concentric channels radiating from a common central area; a plurality of mutually-spaced point incisions arranged in a suitable two dimensional matrix as well as many other patters as envisaged by the man of the art. Some non-limiting examples of incision patterns are illustrated in FIGS. 2A-2F.

As an alternative, the incision may be provided by micro fissuring or micro-punctures. The incisions are performed in such manner that the resulting portions of the cartilage containing tissue remain connected. Such connection may be a portion of the cartilage that is connected to both newly formed segments or that the segments are connected via another component of the cartilage-containing tissue (e.g. bone or artificial or bio-artificial matter). Nevertheless, where the cartilage-containing tissue comprises bone, it is preferred that the cuts would not penetrate the bone portion of the cartilage and that they would be done with sharp knives or blades applying perpendicular pressure and press-fit surgical technique, that may minimize the formation of fibrous cartilage or scar-like tissue. The incisions may be performed by variety of means and in any form, including pins, needle, injection of air or liquid by pressure, pinholes, lines, dashed lines, concentric circles, spiral and any combination thereof (see also FIGS. 2A-2F). The incisions can also be made by using laser beams or any similar method for cutting or making fissures. For the purpose of the present invention, the term incisions, cuts, fissures or micro-fissures will have the same meaning, regardless the mean for achieving such incisions, cuts, fissures or micro-fissures.

In some embodiments, the incisions are made with a fine blade or needle. When the blade is a thin razor blade the mechanical damage of the incision itself may result in cell death of the cells populating 10-30 μm surrounding the cut, or even less (e.g. 1 μm), when using an appropriate tool with extremely fine cutting edge.

In one application of the invention, a balance may be maintained between the amount of viable cells that are rendered so due to incisions (and subsequent cryogenic preservation) and the biomechanical properties of the tissue which may be reduced due the injury of the incisions. Accordingly, the injury level to the tissue should be maintained below a certain threshold or limit in order to maintain a desired balance between the amount of viable cells and the bio-mechanical properties of the cartilage layer. One method of doing so would be a mesh cutting pattern using a single or double comb-like blade head as illustrated in FIG. 10B. As appreciated, the comb-like blade head may be shaped with variable sizes of teeth and gap structures.

It should also be noted that performing incision in the tissue with more than one blade creates pressure on the tissue. It is possible and reasonable that the combination of cutting and pressure applied to the cartilage during cutting has a beneficial effect of reducing the weight of the cartilage containing tissue.

Reducing the weight of the cartilage-containing tissue may be done by any method known in the art to remove components from a tissue without destroying its overall structure in a manner that would prevent its grafting or significantly reduce the chondrocytes' post thaw viability. Non-limiting examples for such methods include applying physical pressure or osmotic pressure, drying, applying a vacuum, an electrical field, a magnetic field, or a chemical gradient. One preferred method of doing so is by performing the said incision as described above. Without wishing to be bound by theory, it is assumed that the weight loss of the cartilage is caused by removal of one or more of the following from the cartilage portion of the cartilage-containing tissue: (a) water, (b) GAGs, (c) proteins. The weight reduction may be for example by at least 1%, 3% or at least 5%. Because they are electrically negatively charged, GAGs or proteoglycans bind to water. Therefore, reducing GAGs or proteoglycans in the cartilage is expected to reduce water in the tissue. It should be noted however, that removal of any of the above (water, GAGs or proteins) can result with non significant or even non-measurable weight reduction and in the context of the present invention it is also regarded as a weight reduction process.

In accordance with another aspect, there is provided a method for providing viable cartilage-containing tissue, comprising:
(a) providing excised cartilage-containing tissue having a cartilage portion;
(b) treating said cartilage-containing tissue by introducing a cryoprotectant agent at least into said cartilage portion; and
(c) cryogenically preserving said treated cartilage-containing tissue under appropriate cryogenic preservation conditions.

According to this aspect, prior to preservation the cartilage containing tissue is treated by the introduction of cryoprotectant agents into the cartilage portion, at least to the intermediate layer of the cartilage containing tissue, if not deeper into the deep layer. This treatment may be done by any method known in the art to introduce components to a tissue without destroying its over all structure in a manner that would prevent its grafting or significantly reduce the chondrocytes' post thaw viability. Non-limiting examples for such methods include immersing the tissue in a cryoprotectant-containing solution, injection, osmosis, applying an electric field, a magnetic field or a chemical gradient, pressure, vacuum, etc. One preferred method of doing so is by performing the incision-providing step as described above whilst the sample is immersed in a solution comprising the cryoprotectant agent or by immersing the tissue, after cutting, in such a solution. Alternatively, the cuts may be performed in dry form or in another solution, after which the cut cartilage is immersed in the cryoprotectant agent containing solution. It being well known that intact cartilage is not permeable to large molecules, it is assumed that the cutting step allows penetration of the high molecular weight cryoprotectant agents, for example in order to replace one or more of the components that are the cause of weight loss.

In an alternative embodiment, the step of cryogenically preserving cartilage may be conducted in such manner that allows control of the ice crystals' propagation and/or morphology so as not to allow planar ice to occupy 20% (either by weight or volume) or more of the cartilage portion of the cartilage-containing tissue. Preferably, planar ice would not occupy 50% or more of the cartilage portion. Preferably substantially no planar ice would be allowed to form (i.e. the cartilage portion would have 0% planar ice). Non-limiting examples for ice morphology-control step include: cutting the cartilage in the manner described above (and by this allowing ice crystals to grow in the cut area), introducing liquid (e.g. water, with or without cryoprotectant agent) into certain areas of the tissue that do not contain cells (chondrocytes), controlling ice crystal morphology by controlling the freezing or vitrification method for example through directional freezing, or by otherwise interfering or perturbing ice crystal propagation by ultra-sound, microwave, electric field, mechanical vibration, introducing chemicals (for example GAGs, proteoglycans), which should cause compartmentalization of the tissue water causing ice crystals to grow only in a desired area etc. This can be also a process of changing the chemical composition or the electrical properties of the ECM in a way that will allow successful cryopreservation, for example, by introducing chemicals or by introducing electrical field or by changing the homogeneous composition of the ECM.

Any of the above methods of the present invention thus relate to providing cryogenically preserved viable cartilage-containing tissue, and may also comprise the step of thawing the cartilage-containing tissue after it has been cryogenically preserved. The cartilage containing tissue after thawing comprises viable tissue, i.e. at least 10% viable chondrocytes throughout the cartilage portion of the cartilage-containing tissue as determined in a live/dead ratio assay.

The thawing may be done in any manner known in the art, such as holding the cartilage-containing tissue at room temperature, submerging the cartilage-containing tissue in a warmed bath, removing the cartilage-containing tissue from the receptacle in which it was frozen and submerging it directly in a container with a solution of a desired temperature (e.g. a solution that that is warmed by being placed in a warmed water bath), using any warming device known in the art such as tube warming blocks, dish warming blocks, thermostat regulated water baths etc.

According to some embodiments of the above methods, the viable cartilage-containing tissue of the present invention comprises a bone segment, and the step of cryogenically preserving the cartilage-containing tissue is preceded with:

providing a pulling member; and

connecting said pulling member to said bone portion.

This pulling member may be used in the thawing step for pulling the partially thawed cartilage from the tube. This may allow immersing the cartilage directly in a solution having a higher temperature than the cartilage, thereby increasing the rate of thawing. Another potential use of the pulling member is that it may be used to secure the cartilage-containing tissue in a test tube before freezing at such position that is above the bottom of the tube (e.g. 1-2 cm above it). This would allow seeding to take place at a part of the solution that does not include the tissue. To that end, the pulling member may be secured to the plug of the test tube. This is achieved by one embodiment in that the puling member is a screw that is connected to the stopper of the test tube and screwed in the bone portion of the cartilage-containing tissue. Alternative embodiments include the use of a vessel which does not require the use of a pulling member, the vessel being described in detailed hereinbelow.

By yet another aspect, the present invention discloses preserved viable cartilage-containing tissue producible by the method of any one of the preceding methods. According to one embodiment, the viable cartilage containing tissue is obtained by the aforementioned methods.

By yet another aspect, the present invention discloses excised cartilage-containing tissue wherein the cartilage portion of the cartilage-containing tissue comprises 2 cuts being up to 1.5 mm apart. For example, the cuts may be as near as 200 μm, 300 μm, 400 μm or even 500 μm apart, and as far as 1.5 mm apart. They may be in form of an array of cuts all being equally distanced one from the other or have different distances.

By another aspect, the present invention discloses thawed viable preserved human cartilage-containing tissue comprising at least 10% viable chondrocytes throughout the cartilage portion of the cartilage-containing tissue after preservation, as tested in the live/dead ratio assay.

By an additional aspect, the present invention discloses excised cartilage-containing tissue comprising live chondrocytes at least 14 days after being excised. In fact, when in a preserved state, the excised cartilage-containing tissue may be maintained (in appropriate storage conditions) for a period longer than 28 days or 45 or even longer than 60 days (theoretically the storage period is unlimited). Such appropriate storage conditions include, in case of freezing or vitrification, temperatures that would prevent thawing of the cartilage or continuous crystallization or recrystallization, preferably such temperatures that are below the vitrification or freezing temperature or the glass transition temperature of the solution in which the cartilage-containing tissue was preserved. Normally such temperature would be below −80° C. or even −196° C. Preferably such cartilage-containing tissue comprises at least 10% live chondrocytes throughout the cartilage portion of the cartilage-containing tissue as assayed using the live/dead ratio, or even at least 50% or more than 80% or even more than 90%.

By still another aspect, the present invention discloses thawed viable preserved human cartilage-containing tissue comprising viable chondrocytes that are at least 50 μm from any edge of the cartilage portion of the cartilage-containing tissue, and even 75 μm, or 100 μm or 150 μm from the edge, or even deeper.

The cartilage-containing tissue of the present invention may be used for any end or purpose known in the art, including especially—for grafting but also for example for storage of cartilage-containing tissue for any other purposed (e.g. extraction of chondrocytes from the thawed tissue). An additional non limiting example for use of the cartilage-containing tissue of the present invention is in the preparation of autologous cartilage implantations (ACI). One option is that the harvested cartilage-containing tissue would be cryogenically preserved per an embodiment of the present invention and later thawed for extraction of chondrocytes that will be used for ACI. Alternatively—a bio-artificial implant comprising chondrocytes may be frozen (and/or thawed) in accordance with the present invention.

When the viable preserved cartilage-containing tissue is to be used for grafting other steps known in the art for grafting may need to be taken, including preparation of the target site to receive the cartilage-containing tissue. The target site of the grafting may be a naturally occurring lesion or fissure or a special cavity produced for the purpose of grafting. One example for the generation of such cavity is the removal of a cylinder or plug comprising cartilage and bone by drilling. Accordingly the shape and dimension of the cavity would be chosen such that the graft may be inserted therein and remain essentially stationary in relation to the graft site after implantation. Normally an osteochondral cylinder for grafting is slightly larger than the target site such that after forced insertion it essentially fills in the cavity and remains practically stationary. Alternatively, a whole condyle or hemi-condyle may be grafted. Grafting or transplantation of a whole condyle or a hemi condyle has the advantage of a uniform surface area and easier surgical technique for the transplanting surgeon. It also has the advantage of replacing large damaged area. The surgical technique of grafting condyle or hemi condyle is well known in the art. Grafting of cartilage-containing tissue in accordance with the invention may be performed in any organ comprising cartilage, for example, ear, nose, or any articular joint, such as knee, elbow, shoulder, hip, etc.

The invention also provides an apparatus for preparing a cartilage-containing tissue for subsequent cryogenic preservation, the apparatus comprising:
a holder for holding said cartilage-containing tissue;
a cutting head comprising at least one incision-forming element for forming an incision in a cartilage portion of said cartilage-containing tissue when held in said holder.

In accordance with one embodiment of the invention, the apparatus is configured to provide incisions of a predetermined depth within said cartilage portion, the incisions in the cartilage portion being as defined above with respect to the methods of the invention.

In accordance with one embodiment, the cutting head of the apparatus is adapted for providing an incision pattern on said cartilage portion comprising a plurality of individual incisions. The incision pattern may vary and include, without being limited thereto, any one of the following patterns when viewed in a direction substantially perpendicular to said cartilage portion:—
a plurality of substantially elongate channels in substantially parallel spaced relationship;
a plurality of substantially elongate channels radiating from a common central area;
a plurality of substantially concentric channels radiating from a common central area;
a plurality of mutually-spaced point incisions arranged in a suitable two dimensional matrix.

In accordance with another embodiment, the holder comprises a cup having a well-shaped cavity for receiving said cartilage containing tissue, and said cup is removably mounted to a table comprised in said apparatus. The apparatus may be configured for traversing the table along a substantially horizontal path such as to enable different parts of the cartilage portion to be aligned with the cutting head.

The invention also provides a vessel for containing the cartilage-containing tissue obtainable by the method of the invention, the vessel comprising:
a substantially impermeable body having a first open end and a second open end at longitudinally opposite ends thereof and defining a containing volume;
a first end plug and a second end plug for reversibly sealing said first open end and a second open end, respectively.

According to one embodiment, the vessel may optionally be tubular, and may comprise a body of a generally uniform cross-section.

According to another embodiment, the body is made from an optically transparent material.

According to yet another embodiment, at least one said end plug of the vessel comprises a graspable portion and a sealing portion. The sealing portion may comprise a stem and a plurality of ribs adapted for sealing with respect to a corresponding said open end when engaged therewith. Alternatively, at least one end plug may comprise a threaded portion adapted for sealing engagement with a complementary-threaded portion comprised in the corresponding open end of the vessel.

In accordance with one embodiment, the first end plug may further comprise an internal anchoring arrangement adapted for facilitating anchoring of the first end plug to fluid material that may be provided and frozen in said containing volume. Without being limited thereto, the anchoring arrangement may comprise a first strip arrangement attached to the first end plug to an inward facing portion of the sealing portion of the first end plug.

According to a further embodiment, the second end plug of the vessel comprises a second strip arrangement comprising a strip of material having a first end attached to an inward facing portion of said sealing portion of the second end plug, and a second free end. The strip preferably has a length sufficient to enable said free end to extend to an outside of the vessel when said second end plug is engaged in a non-sealing manner with respect to said second end. According to one non limiting embodiment, the length of the strip is 2 cm.

According to another aspect of the invention, an end plug for a vessel or the like is provided that facilitates anchoring of the plug to contents of the vessel when this undergoes a cryogenic procedure. Such an end plug of the vessel may comprise a graspable portion and a sealing portion. The sealing portion may comprise a stem and a plurality of ribs adapted for sealing with respect to a corresponding said open end when engaged therewith. Alternatively, such an end plug may comprise a threaded portion adapted for sealing engagement with a complementary-threaded portion comprised in the corresponding open end of the vessel. Such an end plug may comprise an internal anchoring arrangement adapted for facilitating anchoring of the first end plug to fluid material that may be provided and frozen in said containing volume. Without being limited thereto, the anchoring arrangement may comprise a first strip arrangement attached to the first end plug to an inward facing portion of the sealing portion of the first end plug. Alternatively, the end plug may comprise a strip of material having a first end attached to an inward facing portion of said sealing portion of the second end plug, and a second free end. The strip may have a length sufficient to enable said free end to extend to an outside of the vessel when said end plug is engaged in a non-sealing manner with respect to said second end, and thus allow air or other gases, excess fluids etc to be drained from the vessel while the opening is partially sealed. The strip may comprise a weakened portion that is breakable when the free end is jerked, leaving behind a strip portion that may be immersed in the fluid used for cryogenic preservation, and which is used for anchoring therein during the freezing process. Optionally, the portion of the strip that remains in the vessel may comprise barbs, projections, apertures and so on to enhance the anchoring characteristics thereof when the fluid freezes. Thus, according to this aspect of the invention, such an end plug may be used for vessels having a single opening, for example, a test tube, or for multiple openings, for example the vessel of the invention having first and second openings.

The invention also provides a system for providing a cartilage-containing tissue, comprising a device configured for providing a cartilage-containing tissue having a cartilage portion thereon from a donor; and the apparatus as defined above. In one embodiment, the device, which herein is taken to refer to any suitable tool in addition to the regular meaning of device, may be a drilling device adapted for providing a cartilage-containing tissue in the form of a bone plug having a cartilage portion thereon.

In accordance with one embodiment, the system further comprises a trimming device for trimming a length of said bone plug, the trimming device comprising a cavity for accommodating said plug at least when untrimmed, and a slot extending through said cavity located such as to trim the plug in a corresponding manner. In one embodiment, the slot is provided in a direction substantially perpendicular to a longitudinal axis of the cavity.

In accordance with yet another embodiment, the system further comprises a cutting instrument, for example a knife or saw, adapted for cutting through said bone plug accommodated in the cavity while being guided by the slot.

The invention will now be described by way of non-limiting exemplary embodiments. It is to be understood that the scope of this invention should not be construed as being limited to these embodiments and that any combination or permutation of the embodiments is within the scope of this invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Further, although the invention is described in conjunction with specific embodiments thereof, it is evident that many alternatives, modifications and variations will be apparent to those skilled in the art.

All publications, patents and patent applications mentioned in this specification are herein incorporated in their entirety by reference into the specification.

### BRIEF DESCRIPTION OF THE DRAWINGS

In order to understand the invention and to see how it may be carried out in practice, some embodiments will now be described, by way of non-limiting example only, with reference to the accompanying drawings, in which:

FIG. 1 is a schematic illustration in isometric view of a cartilage-containing tissue obtained according to an embodiment of the invention.

FIGS. 2A to 2F are schematic representations in top view of alternative incision patterns that may be provided on the cartilage portion of the embodiment of FIG. 1.

FIG. 3 illustrates in side cross-sectional view the embodiment of FIG. 1 in relation to a body site prepared on the patient that is to receive the cartilage-containing tissue.

FIG. 4 is a schematic illustration of some of the elements of a system for providing a cartilage-containing tissue according to an embodiment of the invention.

FIG. 5 is a schematic illustration in isometric view of a plug cutter drill head according to an embodiment of the invention.

FIGS. 6A and 6B are schematic illustrations, in isometric view and side cross-sectional view, of a trimming device according to an embodiment of the invention.

FIGS. 7A and 7B are schematic illustrations, in top view and side view, of a cutting station which may be employed in the methods of the invention.

FIG. 8 is a schematic illustration in isometric view of a cradle of the embodiment of FIGS. 7A and 7B.

FIGS. 9A and 9B are schematic illustrations, in isometric view and side cross-sectional view, of a cup or holder used for holding a cartilage-containing tissue of the embodiment of FIGS. 7A and 7B.

FIG. 10A is a schematic illustration, in isometric partial view of a cutting head of the embodiment of FIGS. 7A and 7B; FIG. 10B is a schematic illustration in side view, of the blade head of the embodiment of FIG. 10A; FIG. 10C is a schematic illustration in front cross-sectional view, of a variation of the blade head of the embodiment of FIG. 10A; FIG. 10D is a schematic illustration in isometric bottom view, of a variation of the blade head of the embodiment of FIG. 10A.

FIG. 11 is a schematic illustration in cross-sectional side view of a storage vessel according to an embodiment of the invention.

FIG. 12 is a graph showing the effect of cartilage incisions (% of surface injected) on the percentage (%) cell viability (-♦-) or percentage of matrix stiffness (-▪-).

### DETAILED DESCRIPTION OF SOME EMBODIMENTS

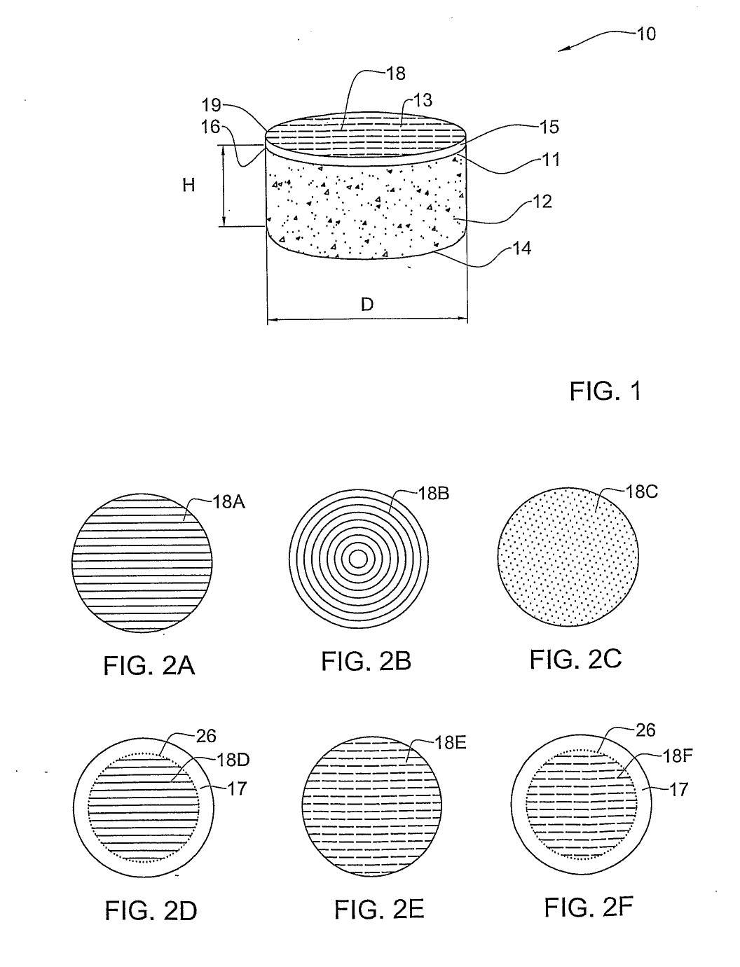

According to the present invention, a cartilage-containing tissue, excised from a suitable donor, may be cryogenically preserved. According to an embodiment of the invention, such a cartilage-containing tissue is in the form of a “bone plug” or “osteocartilage plug”, and is also referred to synonymously as such herein. Referring to FIG. 1, the cartilage-containing tissue or bone plug is generally referenced with numeral 10 and comprises a generally cylindrical substrate 12 of bony tissue or the like topped by a cartilage portion or layer 15.

It should be noted that in other embodiments of the invention, it is possible for the bone plug to be provided in a different form, for example when chiseled or sawn from a donor bone rather than drilled therefrom, and may have any other appropriate shape, for example a wedge, cube, and so on, having a uniform or non-uniform cross-section. In yet other embodiments of the invention, the cartilage-containing tissue comprises the whole hemicondyle or a part of the hemicondyle obtained from a donor. The present invention is applicable to all such embodiments in a similar manner to that described herein for the cylindrical bone plug, mutatis mutandis. In any case, the thickness t of the cartilage layer 15 may be uniform or non-uniform, and the average thickness may vary from donor to donor, or between different parts of the body from which the bone plug 10 may be excised. Typically, the thickness t may be between about 2 mm and about 5 mm in humans, though the invention is in no manner limited by this dimension. At least one, and preferably a plurality of incisions 18 are formed over the surface of the cartilage layer 15, the depth of the incisions typically being, but not limited to, less than the depth t, so that the subchondral layer, i.e. bony substrate 12 is substantially uninjured or unaffected. Thus, it is also possible for the incisions 18 to be formed having a depth greater than t, and also penetrate into the bony substrate 12.

Thus, the incisions 18 increase the surface area of exposed cartilage material, and furthermore provide access channels into the middle cartilage layer from the outside of the cartilage portion.

The incisions 18, also referred to interchangeably herein as cuts, apertures, fissures, and so on, may be arranged in any suitable pattern over the exposed surface of the cartilage layer 15. By way of non-limiting example, and referring to FIGS. 2A to 2F, respectively, the incisions 18 (which also collectively refers to particular configurations of incisions 18A to 18F) may have any one of the following forms:—
(a) a plurality of parallel laterally-spaced linear incisions 18A extending substantially from one part of the edge 19 of the cartilage layer 15 to a longitudinally opposed part of the edge 19;
(b) a plurality of radially-spaced generally concentric circular or elliptical incisions 18B, arranged between the center of the cartilage layer 15 and an outer edge 19 thereof;
(c) a plurality of mutually-spaced point incisions 18C, having any suitable shape such as pinholes or circular incisions, or small linear cuts, for example, and being arranged in a suitable two dimensional matrix over substantially the full surface of the cartilage layer 15;
(d) an arrangement similar to that of FIG. 2A, but wherein the longitudinal extent of each linear excision 18D falls short of the edge 19 of the cartilage layer 15, leaving an annular zone 17 free of incisions circumscribing a central portion 26 that comprises said incisions.
(e) a plurality of parallel laterally-spaced linear arrangements of linearly arranged segmented incisions 18E extending substantially from one part of the edge 19 of the cartilage layer 15 to a longitudinally opposed part of the edge 19; this arrangement is similar to that illustrated in FIG. 2C, with the main difference that the incisions 18E are have a length dimension generally larger than a width dimension over the surface 13 of the cartilage layer 15;
(f) an arrangement similar to that of FIG. 2E, but wherein the longitudinal extent of each line of linearly arranged segmented incisions 18F falls short of the edge 19 of the cartilage layer 15, leaving an annular zone 17 free of incisions circumscribing a central portion 26 that comprises said incisions.

Of course, many other arrangements and patterns of incisions over the surface 13 may also be provided, for example a spiral. By way of example, the width of each incision 18 may be about 15 microns to about 25 micron, or between about 25 micron to about 100 micron, though this width may be greater than about 100 micron or less than about 15 micron, and the length thereof may be from be about the same as the width dimension, and by way of example may range between 0.1 mm (e.g., FIG. 2C) to the full cord dimension of the cartilage layer 15 (e.g., FIG. 2A). The spacings between adjacent incisions, for example between adjacent incisions 18A of FIG. 2A, or between concentric incisions illustrated in FIG. 2B, or between adjacent linear incisions or linearly arranged incisions illustrated in FIGS. 2C to 2F, may be, for example, about 0.4 mm. The depth of the incisions may be substantially uniform across the cartilage layer, or alternatively may vary from incision to incision.

The substrate 12 may be generally cylindrical, and may be geometrically defined in terms of a generally diameter D and height H for convenience; as already mentioned, the plug 10, and thus substrate 12 may have any other convenient shape, which may be defined geometrically according to particular dimensional parameters in a manner best suited thereto, mutatis mutandis. Typical non-limiting values for diameter may range from about 13 mm to about 15 mm, but may be smaller than 13 mm or greater than 15 mm, and may vary from case to case. Typical non-limiting values for the height H may range from 8 mm through 10 mm to about 12 mm, though may be smaller than 8 mm or greater than 12 mm, and may vary from case to case. Alternatively, the substrate 12 may assume a frusto-conical form, having a cross-section of diameter conically tapering between the longitudinally spaced proximal end 11 and distal end 14.

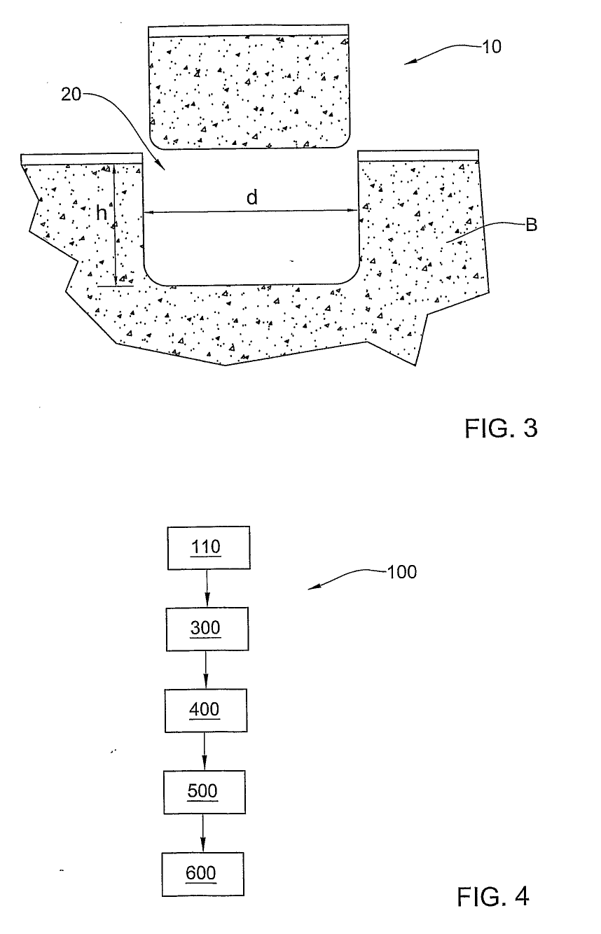

As illustrated in FIG. 3, according to the invention, the bone plug 10 is adapted for insertion into a suitable cavity 20 provided in the patient, which may be a human or a non-human patient (typically after having been frozen and subsequently thawed according to the invention). The cavity comprises an internal diameter d configured for receiving and snugly retaining the plug 10 therein. Thus, where the plug 10 is of generally cylindrical form, the diameter d is slightly smaller than the diameter D of the plug 10. Similarly, where the plug 10 is of generally frusto-conical form, the diameter d corresponds to a diameter of the plug 10, somewhere generally intermediate between its maximum and minimum diameter. Optionally, the cavity may be frusto-conical, tapering in the distal direction into the bony tissue B. The depth h of the cavity 20, taken from the upper extent of the bony tissue B, is also such as to accommodate the height H of the plug 10, and is thus h has substantially the same dimension as H, or in some cases h may be slightly deeper than the dimension of H to allow some clearance.

Optionally, the distal end of the plug 10 facing the cavity 20 may be chamfered or beveled (not shown) to facilitate insertion of the plug 10 into the cavity 20.

According to the invention, and referring to FIG. 4, a system, generally designated with the numeral 100 is provided for excising, preparing and dealing with the bone plug 10 until required for use with a patient.

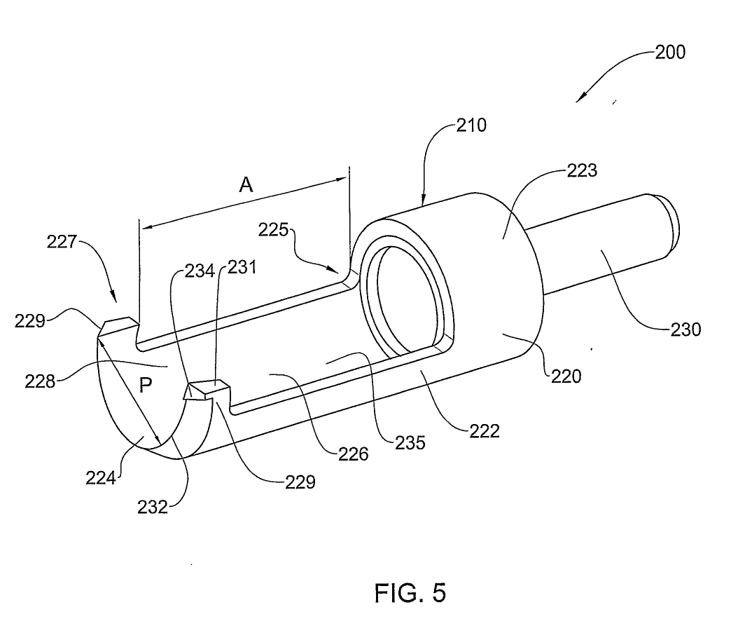

The system 100 may comprise a drilling device 110 having a plug cutter drill head 200 adapted for cutting an undressed or untrimmed bone plug 10′ from a donor, which may be a cadaver bone comprising a suitable cartilage layer thereon, for example. The untrimmed bone plug 10′ is axially longer than the trimmed bone plug 10 that is to be inserted into cavity 20 after trimming, as will be described in greater detail. Referring to FIG. 5, a particular embodiment of the drill head 200 comprises a generally cylindrical stepped body 210 having a generally cylindrical hollow bit 220 coaxial with shank 230 that is reversibly attachable to the drilling device 110 via a chuck or the like, for example. The bit 220 comprises a generally cylindrical wall 222 having a closed proximal end via wall 223 attached to or integral with said shank 230, and an open distal end 224. A lateral plug retrieval opening 225 provides communication between the interior 226 of the bit 220 and the exterior thereof, and is of a size sufficient to allow extraction of the plug 10′ therefrom. Accordingly, the axial dimension A of opening 225 is generally greater than the height of the untrimmed bone plug 10′, and extends circumferentially to an angular extent at least 180° or greater. Alternatively, the axial dimension A of opening 225 may be generally less than the height of the untrimmed bone plug 10′, and/or extends circumferentially to an angular extent less than 180°, in which case the plug 10′ can be axially retrieved via opening 224. In any case, the axial extent of the interior 226 is sufficient to accommodate a desired height of untrimmed bone plug 10′.

A slotted cylindrical wall segment 227 is provided between opening 225 and distal end 224, defining axial slot 228 and arcuate horns 229 on either side thereof connected to cylindrical wall 235 that extends from proximal end wall 223 to distal opening 224. Slot 228 may have any suitable angular extent, which may range, for example, between about 30° and about 60°, through about 45°. The distal opening 224 comprises an annular cutting edge 232 that is beveled for facilitating rotational cutting, the annulus being interrupted by said slot 228. Furthermore, at least one of said arcuate horns 229 comprises a generally axially aligned beveled cutting edge 231, and axially protruding cutting tooth 234.

The internal diameter of the cutting edge 232 is such as to provide a bone plug having diameter D. The cutting edge 232 of the drill head 200 is sufficiently sharp such as to enable the same to penetrate the relatively soft cartilage layer 15 by simply axially pressing the drill head 200 into the same. This procedure serves to stabilize the position of the drill head on the cartilage layer 15, as the drill head begins to rotate and penetrate into the bony layer 12, when the cutting tooth 234 acts thereon to cut the plug 10.

The drill head 200 is thus adapted for providing substantially cylindrical untrimmed bone plugs 10′. In variations of this embodiment, the interior 226 may be suitably tapered to enable frusto-conical shaped bone plugs to be cut from the donor.

The drill head 200 may be made from any suitable medically compatible material, for example any suitable stainless steel such as stainless steel 420.

In use, the drill head 200 is connected to the drilling device 110, and is brought into contact with a suitable bone tissue of the donor. As the drill head is rotated, an untrimmed bone plug 10′, having a layer of cartilage thereon, is cut from the bony tissue, and then the untrimmed bone plug 10′ is removed from the drill head 200, either via the side opening 225 or via the distal opening 224.

Alternatively, any other suitable plug cutter drill head for providing the plug 10. Alternatively, any other appropriate device, including suitable tools, for example saws, wires, chisels, scalpels and so on, may be provided in place of the drilling head, for providing a cartilage containing tissue of the desired shape and size.

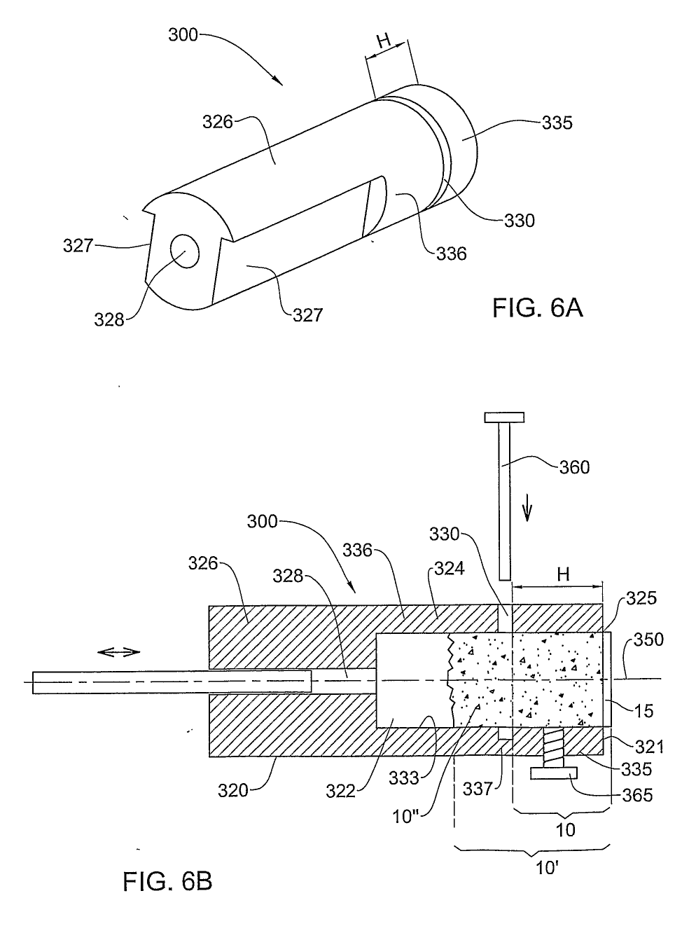

According to the invention, and as illustrated in FIGS. 4 and 6A to 6B, the system 100 may comprise a trimming device 300 (also referred to herein as a graft sizing device or GSD) for trimming the untrimmed bone plug 10′ to the required axial dimension or height H, as required for eventual insertion into a standard-sized well or cavity 20. The trimming device 300 comprises a body 320 having a mitre box cylindrical fore section 324 and a coaxial graspable aft section 326. Fore section 324 comprises an opening 325 at a proximal end 321 leading to cavity 322 of diameter and depth sufficient to permit the untrimmed bone plug 10′ to be accommodated therein in a reasonably tight manner, such that it does not freely rotate therein, or at least may not slide out therefrom under gravity, but on the other hand does not provide undue resistance when pushed therefrom from the inside, as will become clearer herein. Alternatively, the diameter of cavity 322 provides a small clearance with respect to the diameter of the plug 10′, so that it is able to slide freely therein, and a locking arrangement, for example in the form of a radial tightening screw 365 that may be reversibly turned towards the plug 10′ may be used for retaining the same in place. While the cavity 322 is of cylindrical cross-section in this particular embodiment, it may also be used for holding and trimming bone plugs that are not of circular cross-section, so long as they fit therein. Further, in other embodiments, it is also possible for the cross-section to be of any other shape, for example oval, polygonal, etc., and relatively uniform or non-uniform along the axial length of the cavity—for example the cavity 322 may be frusto-conical. Aft section 326 comprises substantially parallel graspable surfaces 327 on the outside thereof, and a rectilinear and typically axial lumen 328 extending from the aft end 329 of the body 320 to the internal cavity 322. A circumferential slot 330 is provided in the fore section 324, radially extending through the wall 332 thereof around the full circumference of the interior surface 333 of cavity 322, and thus dividing the fore section 324 into a fore part 335 comprising opening 325, and an aft part 336 joined to said aft portion 326, via a bridge 337. The slot 330 is aligned on a plane substantially perpendicular to the axis 350 of cavity 322, and is of an axial width sufficient to permit insertion of cutter 360, which may be a saw blade, for example. Further, the slot 330 is axially spaced by a distance H from the proximal end 321.

Optionally, a plurality of axially spaced slots 330 may be provided, each at a particular axial distance from proximal end 321, to enable the trimming device to be used for trimming bone plugs to different desired sizes.

In operation, an untrimmed bone plug 10′, for example obtained with the aid of drill head 200, is inserted into the cavity 322 such that the cartilage layer 15 is projecting from the proximal end 321, and thus the interface 16 between the bony tissues 12 and the cartilage layer 15 is axially aligned with the proximal end 321. The trimming device 300 is then grasped by a suitable clamp via surfaces 327, and cutter 360 is aligned with slot 330, cutting or sawing the untrimmed bone plug 10′ along a plane defined by the slot 330, into a trimmed bone plug 10, and a bone fragment 10″. At the end of the cutting or sawing process, the cutter 360 is removed, and a rod 370 is inserted into cavity 322 via lumen 328, pushing out the bone fragment 10″ and bone plug 10, which has the required axial dimension H.

The trimming device 300 may be made from any suitable medically compatible material, for example any suitable stainless steel such as stainless steel 420.

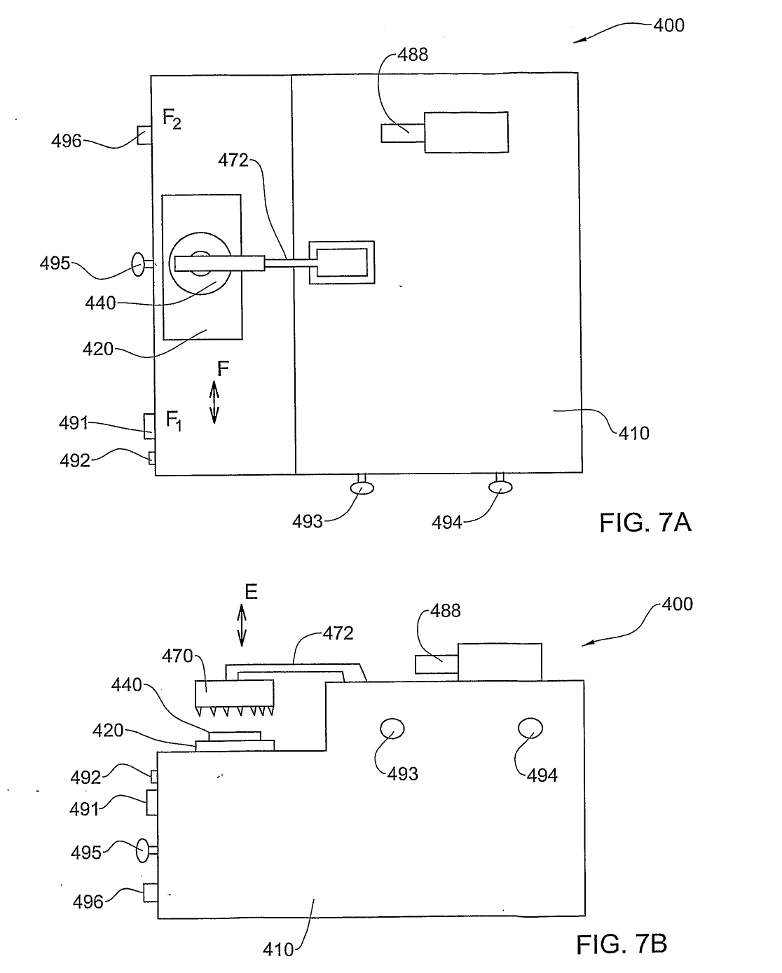

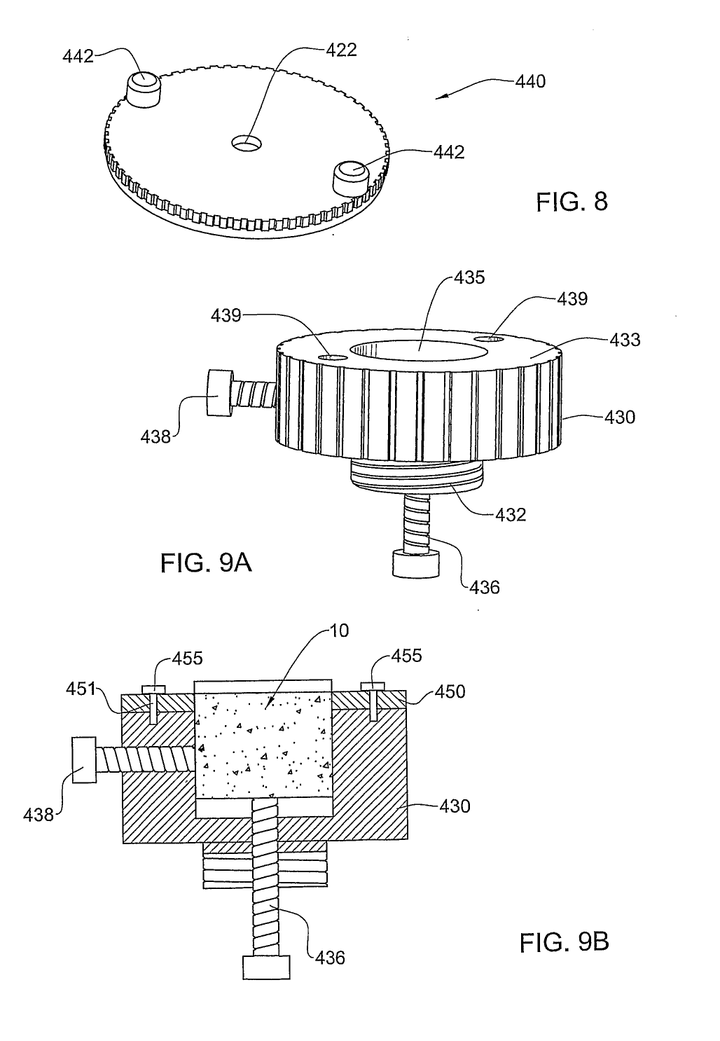

According to the invention, and also referring to FIGS. 7A, 7B, 8, 9A. 9B the system 100 comprises a cutting station 400 (also referred to herein as a cartilage preparation device or CPD), an apparatus or device for applying said incisions 18 to the cartilage layer 15. In this embodiment, the cutting station 400 is configured for powered as well as for manual operation, and comprises a casing 410 having a substantially horizontal table 420 that is reciprocably moveable along direction F by means of a suitable mechanism (not shown) in said casing 410. The cutting station 400 comprises a suitable power source (not shown), for example electrical batteries, or alternatively may be connected to an electrical power source such as an electric mains for example. A disc-shaped cradle 440 (FIG. 8) comprises an externally-threaded projection (not shown) at an underside thereof, which enables the cradle to be securely engaged to the table via complementarily threaded well comprised in the table. The cradle 440 comprises handles 442 for facilitating rotating the of cradle with respect to the table to engage/disengage one with the other, and further comprises an alignment well 422 for receiving and engaging with the projection 432 of cup 430 (FIGS. 9A, 9B), for example by means of a complementaty thread arrangement, bayonet arrangement, and so on. Alternatively, the disc-shaped cradle 440 (FIG. 8) may be adapted for allowing the cradle to be rotated about an axis perpendicular to the table to any desired angular orientation with respect to the table and is also reversibly mountable to the table 420. The cup 430 comprises a well-shaped cavity 435 for receiving and accommodating a trimmed plug 10, such that the cartilage layer 15 protrudes from the lip 433 of the cup 430. Accordingly, the depth of the well cavity 435 is typically designed to be substantially equal to H. Optionally, an axially adjustable screw arrangement 436 may be provided co-axially with said projection 432 and passing therethrough to cavity 435 for adjusting the position of the cartilage layer 15 with respect to said lip 433. Further optionally, the diameter of cavity 435 provides a small clearance with respect to the diameter of the plug 10, so that it is able to slide freely therefrom after being prepared by the station 400, and a locking arrangement, for example in the form of a radial tightening screw 438 that may be reversibly turned towards the plug 10, may be used for retaining the same in place. Further optionally, and referring to FIG. 9B, one or more spacer annular discs 450 may be provided for mounting onto lip 433 to effectively raise the height thereof, and thus enable plugs 10 having a height H greater than the depth of the cavity 322 to be accommodated therein, while aligning the interface 16 with the upper surface of the disc 450. The discs 450 may be secured to the lip 322 via bolts 455 or the like, for example that pass through suitable apertures 451 through the disc and into aligned recesses 439 in the cylindrical wall of the cup 430.

The cutting station 400 further comprises a cutting head 470 comprising at least one incising or cutting means for producing one or a plurality of desired incisions 18 over the cartilage layer 15. The cutting head 470 is vertically aligned with the table 420, and more specifically with a zone of the cradle 440, and is mounted at the end of an arm 472 cantilevered from a drive unit (not shown) that is adapted for providing a reciprocable movement (for example linear or arcuate) to the head along general direction E substantially orthogonal to the table or cradle.

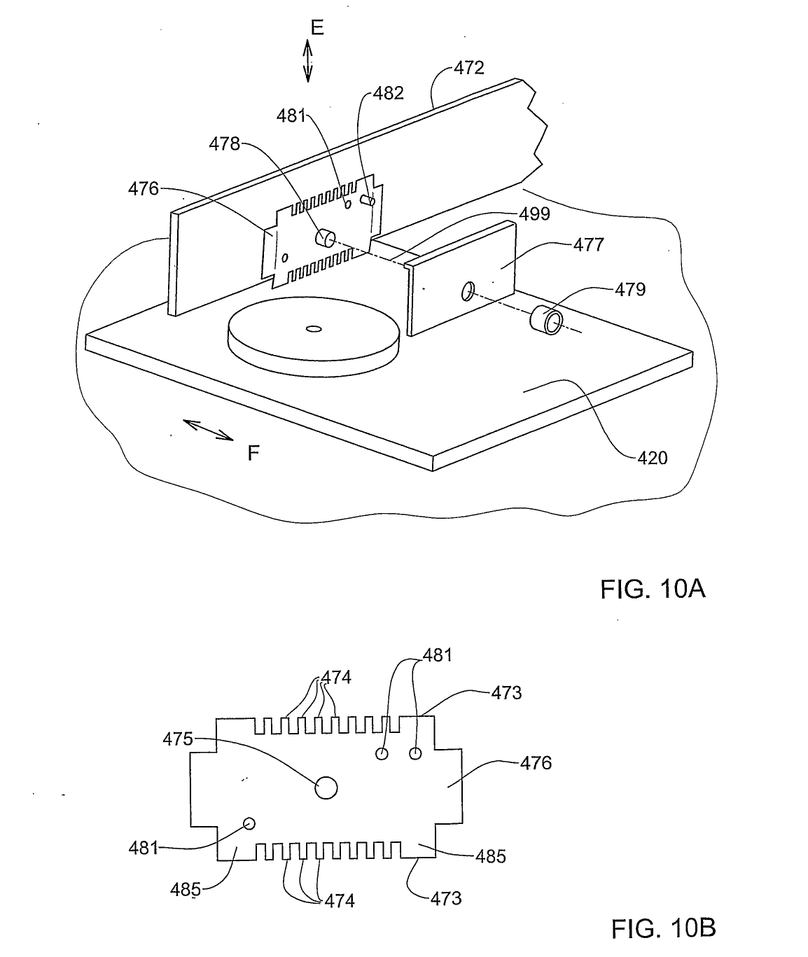

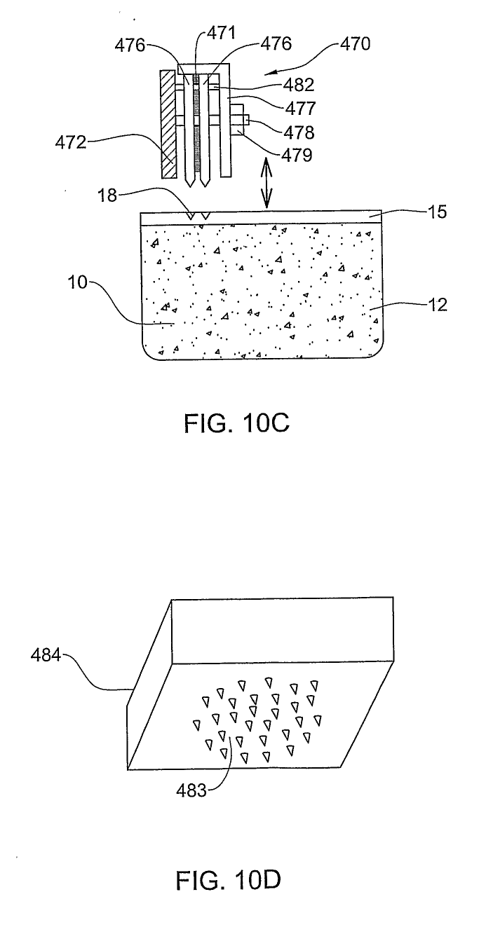

Referring to FIGS. 10A to 10C, in one particular embodiment of the invention, the cutting head 470 comprises at least one generally rectangular cutting blade 476 having a pair of comb-like cutting edges 473 along either longitudinal sides thereof, defining a plurality of longitudinally aligned and spaced cutting projections or elements 474. Each projection or element 474, when inserted into the cartilage layer 15, for example as the arm 472 is moved in a downward motion in direction E towards the table 420, produces therein an incision 18 of comparable cross-sectional shape and size. Thus, when the full cutting edge 473 is brought to bear against the cartilage layer 15, an incision pattern complementary to the cutting edge 473 is formed therein. At least one aperture 475 enables the blade 476 to be mounted to a corresponding threaded stud 478 on arm 472, and an L-shaped shield 477 is clamped to the blade 476 and arm 472 with nut 479, such that cutting projections or elements 474 protrude beyond the arm 472 and shield 477 towards the table 420. Further, the capability for lateral movement of table 420 in direction F enables the relative alignment between the cutting edge 473 and the cradle 440 to be varied in a continuous or stepped manner. Thus, when a plug 10 is properly accommodated in a cup 430 mounted on cradle 440 and table 420, any desired incision pattern may be produced on the cartilage layer 15 by repeatedly bringing the cutting edge 473 into cutting contact with the layer 15, each time laterally moving the table 420 or rotating the cradle 440 with respect to a previous position. In other embodiments, the table and/or cradle may be fixed, and the cutting head is moved across the cartilage layer 15. When the cutting edge 473 becomes blunt, or whenever desired, the blade 476 may be removed, rotated 180° and remounted such as to expose and enable the other cutting edge 473 to be used.

Optionally, the cutting head 470 may comprise a plurality of said blades 472 in substantially parallel and optionally staggered relationship, secured in a similar manner to that described for the single blade, mutatis mutandis, such as to produce a corresponding plurality of linearly aligned incisions. Further optionally, the spacing between blades 476 may be adjusted by using spacing elements 471 between adjacent blades. The blades 476 may optionally comprise a plurality of axially spaced alignment apertures 481 that enable the blades to be aligned one with the other in different staggered relationships by selecting the particular aperture 481 for each blade through which an alignment pin 482 (mounted on arm 472) is to be passed. In this manner, it is possible to provide a greater number of incisions for each reciprocation cycle of the arm 472.

Alternatively, and referring to FIG. 10D, for example, the cutting head 470 may comprise a block 484 having a plurality of small blades 483, needles or the like, arranged in any desired arrangement such as to provide the full incision pattern over the cartilage layer 15 in a single reciprocation cycle of the arm 472, as the block is stamped over the layer 15.

In each case, a stopping means may be provided to limit the penetration of the cutting edges into the cartilage layer 15. For example, referring to FIG. 10B, the blades 476 comprise longitudinal abutting portions 485 that are adapted for abutting against lip 322 (or the upper exposed annular face of spacer disc 450) to prevent further movement of the blade. Since the lip 322 is aligned with interface 16, penetration of the cutting edges into the bone layer 12 is substantially prevented.

The cutting station 400 preferably also comprises a suitable mechanism (not shown) for adjusting and controlling the lateral travel of the table between each action of the head 470, and this may be controlled by means of a control such as an adjustment micrometer 488. On the other hand, the depth of the incisions, i.e., the travel of the head 470 in direction E may be controlled by controlling the force of the head 470 when this abuts the plug 10, and may be controlled by means of blade force knob 496 and speed control knob 494 (see below).

Referring again to FIGS. 7A, 7B, the cutting station 400 comprises a suitable user interface to enable and facilitate operation of the apparatus. The user interface may comprise the following:—
an ON/OFF switch 491;
a reset switch 492 for initiating the cutting sequence after all settings of the different switches and knobs have been performed;
a speed control knob 494 for controlling the speed of cutting of the head 470, i.e., the reciprocation cycle time of the head 470, which is also a measure of how quickly the cartilage layer 15 is being processed (this is also a function of how many passes the head 470 needs to make with respect to layer 15, which in turn depends on the incision pattern desired, and the particular configuration of the head 470
manual operating knob 493, which allows manual operation of the device; the knob 493 allows the head 470 to be brought into incision contact with the layer 15 manually, and thus the knob 470 is mechanically coupled to the actuating mechanism of the head 470;
table release knob 495, which may be used to lock the table 420 when mounting the cradle 440 thereto and/or adjusting the position of the cup 430 or head 470 with respect thereto; the knob 495 may then release the lock on the table 420 so that it may move along direction F;
blade force knob 496, which controls the force that the head 470, and in particular of the individual cutting elements 474, brings to bear against the cartilage layer 15.

Operation of the cutting station 400 may be as follows. The table 420 is prevented from moving via knob 495, and the cradle 440 is mounted to the table 420. A trimmed plug 10 is accommodated in the well 435 of a cup 430, such that the interface 16 is substantially aligned with lip 433 of the cup, using screw arrangement 436 if the height H of the plug 10 is smaller than the depth of the well 435. Alternatively, if the height of the plug 10 is greater than the depth of the well, one or more discs 450 may be used, and the interface 16 aligned with the upper surface thereof, and the screw arrangement 436 may also be used for fine adjustments if desired. Similarly, if it is desired to limit the depth of the incisions 18, the position of the interface 16 may be set to be below the lip 322 (or below the level of the upper surface of disc 450). The cup 430 is then mounted onto the cradle 440. Preferably, the precise orientation of the blades (for example about axis 499 through the center of the blades 476) is first set such as to provide alignment with the cartilage layer 15, and in particular the interface 16. This alignment may be carried out in a pre-operation, by loosening the blade(s) 476 with respect to arm 472, and gently resting the cutting edge of the blade(s) on the lip 322 (or of the upper surface of the uppermost disc 450 mounted thereon), with an empty cup 430, i.e., prior to inserting the plug 10 therein, and the blade(s) fixed in position by means of screws 479. Once the alignment calibration is complete, the plug 10 may be accommodated in the cup 10, as before. Then, the speed, cutting force and lateral step dimension (in direction F) are set to provide a desired depth and pattern of incisions, and the cutting operation may begin. In the cutting operation, the table 420 is moved to its furthest position along direction F (to the left extremity F1, for example, or alternatively to the right extremity F2), such that the cutting elements 474 are aligned with a corresponding extremity of the plug 10 (or with a location on the table beyond the plug) when viewed from above. The cutting station 400 is switched on to activate the head 470 which in once reciprocation cycle brings the cutting elements 474 into cutting contact with the cartilage later 15 such as to produce one or more rows of incisions 18 thereon, and then moves away the head 470 from the plug 10. The table 420 is moved along direction F towards the other extremity (towards the right F1 in the above example) in increments and at each step increment a reciprocation cycle is applied to head 470, thereby producing another row or another set of rows of incisions 18 on the cartilage layer 18. On completion of the sweep across the layer 15, the station 400 may be switched off, and the cup 430 removed therefrom to enable further processing of the prepared plug 10.

Optionally, an amount of freezing solution, such as for example ethylene glycol solution, or a cryoprotectant solution containing about 5%, 10% or more ethylene glycol or DMSO or any other cryoprotectant used in the freezing process as mentioned in the summary above, may be applied to the cartilage layer 15, and in particular introduced therein via said incisions 18.

In variations of this embodiment, a suitable feedback arrangement may be used for controlling the depth of the incisions 18, and particularly for minimizing or preventing damage to the bony substrate 12. In such cases, a suitable sensor may be provided for sensing the resistance of the plug 10 to the force provided by the head 470, for example by measuring the resistance to movement of the head 470, so that while resistance is within a certain threshold, characterizing penetration through the relatively soft cartilage layer 15 the force is maintained, but when the resistance sharply increases, such as when the head 470 encounters the relatively harder bone material, the head 470 automatically moves away from the plug 10.

According to the invention, any suitable cutting station may be used for providing the incisions 18, and is not limited to station 400 as disclosed herein. According to the invention, the cutting station may comprise any suitable holder for holding the cartilage-containing tissue, and a cutting head comprising at least one cutting element for cutting a plurality of incisions in the cartilage portion of the cartilage-containing tissue when held in the holder.

According to the invention, the system 100 may further comprise at least one vessel or storage container 500 for storing the plug 10 therein, in particular during freezing thereof, in the frozen state after the freezing operation is completed, and optionally for at least part of the thawing thereof. In this embodiment, the container 500 comprises a generally cylindrical body 510 having first and second open ends, 512, 514, respectively, at opposite longitudinal ends thereof, and defining an internal cavity 520. The internal diameter Q of at least a part of the body 510, and thus of one of the open ends, is generally sufficiently larger than that of the plug 10, so as to enable the plug to be inserted and retrieved therefrom without resistance. Accordingly, the diameter Q is greater than diameter D by any suitable margin or radial tolerance r. In some variations of the disclosed embodiment, the tolerance r may be set to be such as to avoid the possibility of the plug 10 rotating about a plane aligned with its longitudinal axis and perhaps getting stuck inside the cavity 520. The body 510 may be made from a optionally transparent material that is preferably not damaged by a freezing process, or alternatively may be made from an optically opaque or translucent material, optionally comprising a window to enable the contents thereof to be viewed from outside. Two stoppers or end plugs 530A, 530B, referred to collectively as 530, are provided for reversibly sealing the ends 512, 514.

Each end plug 530 comprises a sealing portion 540 and a grasping portion 550, coaxially joined or integrally formed one with the other. The sealing portion 540 comprises a central stein 542 and one or a plurality (two shown in the figure) of sealing rings, discs or circumferential ribs 544 (joined or integrally formed with the stem 542) for sealing against the internal wall 522 of body 510. In one of the end plugs 530A, an outwardly bowing strip 546A is provided having ends 547A joined to or integrally formed with the inner facing rib 544. The strip 546A acts as an anchor, securing the plug 530B in place when the freezing solution freezes, and thus preventing the plug 530B from being pushed out if the freezing solution expands during the freezing process. In the other end plug 530B, another strip 546B is provided having one end 547B joined to or integrally formed in a rather loose manner with the inner facing rib 544, and the other end 547C being free. The grasping portion 550 comprises an end disc or plate 555 for abutting against the corresponding edge 552, 554 of the ends 512, 514 respectively, and a graspable portion 556 projects from the plate 555 terminating in another disc or plate 557. A label 559 may be provided on the graspable portion 556, and/or elsewhere on the container 500, comprising any bar code and/or alphanumeric characters, symbols, color codes and so on, such as to convey particular or desired data or other details typically relating to the contents of the container 500.

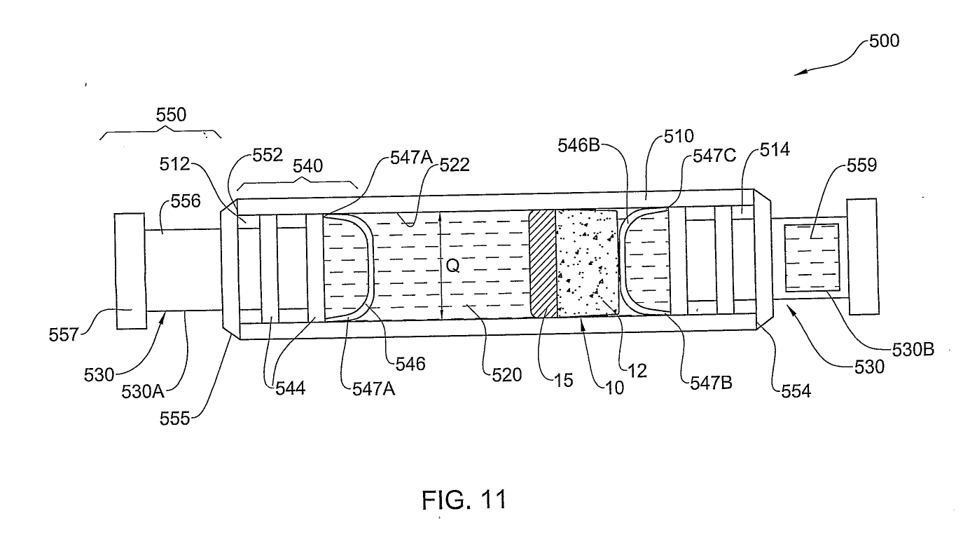

As illustrated in the FIG. 11, said body 510 is of generally cylindrical and uniform cross-section along the longitudinal length thereof. However, in other variations of this embodiment, other cross-sectional shapes for the tubular body may be provided, for example polygonal, oval, and so on. Accordingly, the term “tubular” is herein taken to include, in addition to circular, any other suitable cross-section for the body. Further, it is possible for the two open ends to be of different shapes and/or sizes, and the open ends of the body may optionally be larger, or smaller, than the central section thereof where the plug is accommodated, so long as it is possible to insert and remove the plug 10 with respect thereto. For example, the body may be of frusto-conical shape, having a larger open end for inserting and removing the plug, and a smaller end for enabling a rod or other pushing tool to enable the frozen contents of the cavity 520.

The two end plugs 530 may be substantially identical, or may differ one from the other. For example, the plugs may be color coded, one being red and the other blue. Alternatively or additionally one or both plugs may have a different construction as known in the art, and essentially any type of plug design that is known for reversibly sealing a cylindrical container at one end thereof may be suitable for each end 512, 514.

In use, the container may be sealed in a vacuum bag (not shown) after the freezing process for subsequent cryogenic storage.

Optionally, one or a plurality of axially-extending ribs may be provided in the internal wall 522. Such rib(s) may extend along the full axial length of the body, optionally not including the parts thereof occupied by the sealing portion 540. Alternatively, the rib(s) may extend along a part of the axial length for example in registry with the intended location of the bone plug, and may assist in retaining the plug in place if desired, and also provides a lateral spacing between the plug and the wall 522 which can be filled with freezing solution and possibly enhance the freezing process.

The container 500 may be used as follows. When needed, one of the end plugs 530B is removed, and with the container oriented generally vertically or inclined to the vertical, with the second end plug 530A lowermost, a suitable solution, such as for example a freezing solution such as ethylene glycol solution is injected or otherwise placed in the cavity 520, followed by the prepared plug 10 containing the desired incisions 18. The plug 10 is inserted such that the cartilage layer 15 is facing the lower end plug 530A, and generally immersed in the liquid. The end plug 530B is replaced, taking care to extract excess air in the cavity 520, which may be done by threading the free end 547C of strip 546B between the inner wall 522 and the rings 544 and to outside the container 500, enabling air to escape via the passage formed there; excess freezing solution may also be bled via the same passage. After all the excess air and excess freezing liquid is removed, the strip 546B is torn away from the end plug 530B and removed therefrom via the free end 547C, thereby sealing off the opening 514.

In one method of use, the container 500, containing the plug 10 and solution, may be temporarily stored in a refrigerator at about 4° C. for a time period, for example between 45 minutes and 2 hours.

Additionally or alternatively, the containers may be seeded prior to their introduction to the cryogenic unit 600 (see below). This can be done by briefly by dipping the container 500 in a liquid nitrogen vacuum flask (also referred to herein as a “thermos”) for a short period, say about 5 seconds or up to about 30 seconds, for example, and then this is followed by cryogenic freezing and storage as will be explained below.

At some point after freezing and storage of the container 500 including the plug 10, it may be desired to recover the plug 10 for use with a patient. For this purpose, plug 10 may be thawed using any suitable procedure. For example, the container 500 is removed from the liquid nitrogen thermos and left to stand for a short period of a few minutes, for example 3 minutes, during which time the container may be removed from it vacuum bag. The container is then held in a water bath or the like at a temperature of about 50° C. for a period of about 11 seconds, and during this time the container 500 may be moved around in the bath. The container 500 is then removed from the water bath, the two end plugs 530 removed, and the frozen contents, comprising a frozen cartilage-containing bone plug of solution plus the plug 10, is removed from the cylinder 510. The frozen solution at either longitudinal end of the plug 10 is cut off with a knife, for example, and the plug 10 is held in a tube of PBS (Phosphate Buffered Saline solution) heated to about 40° C. for about 25 seconds. The thawed plug 10 may then be placed in a tube or container having 5% dextrose and 0.9% NaCl for about 5 minutes, after which the plug 10—is transferred to a second container containing 5% dextrose and PBS for another period of about 5 minutes, and then repeated with a third container or tube containing about 5% dextrose and 0.9% NaCl for a final period of about 5 minutes.

According to the invention, the system 100 further comprises a cryogenic unit 600 for freezing the cartilage plug 10 after is has been prepared at the cutting station 400 and sealingly enclosed in the container 500. Such a cryogenic unit 600 may be similar to and operate in a similar manner to that disclosed in U.S. Provisional application No. 60/600,804 and PCT IL 2005/000876, assigned to the present Assignee. The contents of these references are incorporated herein in their entirety.

Alternatively, the cryogenic unit 600 may comprise a set of cooling blocks with channels through which the containers 500 and a reference tube are propelled until they come to rest at a collection block. The movement of the containers 500 through the cooling blocks, in particular the speed therethrough and temperature conditions, is carefully controlled, resulting in a predefined cooling rate. Typically, the containers 500 are placed into the channels following seeding. After reaching the collection box they can be transferred to a deep freeze facility, such as for example a liquid nitrogen thermos. A suitable method and apparatus for this form of cryogenic freezing is disclosed in co-pending PCT application WO 2005/032251, based on U.S. priority application Nos. 60/509,546 and 60/536,508, assigned to the present Assignee. The contents of these references are incorporated herein in their entirety.

### EXAMPLES

### Cartilage Preparation and Protocols

### Materials

Unless specifically said otherwise, materials were obtained as follows: Sucrose S-5016 and Ethylene Glycol E9129/L (Sigma, Israel) F12 medium-01-095-1A, PBS and Penicillin-Streptomycin-Nystatin solution 03-032-1B (Biological Industries, Israel). Viability was tested using live/dead fluorescent dyes (SYTO-13/Propidium Iodide (PI), Molecular probe, USA, according to the manufacturer's manual).

### Handling and Receipt of Human Knee Joint

Human knee joints were provided from cadaver donors by DIZG German Institute for Cell and Tissue Replacement, Berlin, Germany, after being tested for HIV (Human Immunodeficiency Virus), HBV (Hepatitis B Virus) and HCV (Hepatitis C Virus). The knee joints were packaged in RPMI 1640 storage medium (Biological Industries, Israel Cat#01-104-1, [Moore, G. E., Gerner R. E. and Franklin, H. A. (1967) Culture of Normal Human Leucocytes. JAMA 199, 519-524]) containing antibiotics and antimycotics and shipped in ice at a temperature range of 0° C. to 4° C. Upon receipt of the joints a small slice of cartilage was taken to determine cartilage viability before freezing.

In the following examples utilizing human cartilage articular Cartilage, collected from 4 cadaveric human tissue donors aged (43, 18, 25, 30) (8 knees) was harvested by the western Hungarian regional tissue bank in Gyor, Hungary. Harvesting was performed up to 12 hours after death. Processing of the cartilage for freezing initiated between 36-48 hours after death. Specifically, all manipulations of tissue samples were done in a sterile manner. Osteochondral bone plugs in the form of cylinders, 13 mm in diameter, were drilled from human knee condyle using a power surgery drill (Imex, Veterinary Inc. Texas, USA). Harvested cartilage-containing bone plugs were maintained in a buffered physiological solution containing 0.9% NaCl (Sigma, St. Louis, USA) and 1% antibiotics (Penicillin/Streptomycin/Nystatin, Biological Industries, Beit Haemek, Israel) until completion of harvesting.

### Harvesting and Maintenance of Sheep Knee Joint

Fresh cadaver sheep legs were purchased from a slaughter house (Holon Slaughter house, Israel), and all manipulations of tissue samples were done in a sterile manner. Osteochondral bone plugs in the form of cylinders, 13 mm in diameter, were drilled from sheep knee condyle using a power surgery drill (Imex, Veterinary Inc. Texas, USA). Harvested cartilage-containing bone plugs were maintained in a buffered physiological solution containing 0.9% NaCl (Sigma, St. Louis, USA) and 1% antibiotics (Penicillin/Streptomycin/Nystatin, Biological Industries, Beit Haemek, Israel) until completion of harvesting.

### Harvesting and Maintenance of Porcine Knee Joint

Articular Cartilage collected from 20 porcine hind legs harvested immediately after slaughter, was transferred to the processing laboratory for cryopreservation and analysis. Cryopreservation was performed using a directional freezing system. The harvested cartilage-containing bone plugs were maintained in a buffered physiological solution containing 0.9% NaCl (Sigma, St. Louis, USA) and 1% antibiotics (Penicillin/Streptomycin/Nystatin, Biological Industries, Beit Haemek, Israel) until completion of harvesting. Thirty 15 mm cylindrical grafts were examined for cell viability and cell density using fluorescent and confocal microscopy and proteoglycan synthesis via ³⁵SO₄ uptake. Biomechanical assessment was performed on a second set of 9 grafts to determine the matrix instantaneous modulus of elasticity.

In the following, the same procedures were used for both porcine, human and sheep cartilage, unless otherwise specifically noted.

### Maintenance of Osteochondral Cartilage-Containing Bone Plugs (Sheep, Porcine or Human)

The bone plugs, referred to interchangeably herein also as cylinders, were placed in plastic storage containers (the term “container” may be used herein interchangeably with the terms “vessel” or “tube”) containing a solution of Phosphate buffered Saline (PBS) with antibiotics and antimycotics added. Cartilage-containing bone plugs were held in this solution for up to 2 hours until other cartilage-containing bone plugs were harvested. After harvesting of all cartilage-containing bone plugs they were transferred to a storing solution containing an F12 nutrient mixture with antibiotics and antimycotics in disposable 50 ml storage tubes (Corning Incorporated). The cartilage-containing bone plugs were held at 4° C. in refrigeration until freezing, but for no more than 1 week.

### Cartilage-Containing Bone Plug Preparation for Freezing

(1) The cartilage-containing bone plugs were completely immersed in sterile freezing solution (10% EG in PBS) with the cartilage portion facing up. The cartilage was incised using a cutting system comprising a cutting station as shown in FIGS. 7A and 7B using cutting patterns as illustrated in FIG. 1A or in FIG. 1E, which was brought down on the cartilage section of cartilage-containing bone plug such that the blades enter the cartilage from directly above and cut down in parallel cuts to the level of the bone. After cutting, a screw was inserted 3-5 mm into the bone portion of each cartilage-containing bone plug. This screw was attached to a string. Alternatively it may have been attached directly to the stopper of the test tube. The screws did not penetrate the cartilage layer.

The cartilage-containing bone plugs with screw attached were each placed in a separate 16 mm glass freezing tube with the cartilage portion facing the bottom of the tube. Using the string and/or the stopper of the test tube, the cartilage-containing bone plugs were secured such that the cartilage edge of the cartilage-containing bone plug was about 50 mm above the bottom end of the tube. Alternatively this may be done by other methods such as a screw attached to the tube stopper.

Freezing solution was added to the test tube or double-open ended container, similar to container 500 described herein with reference to FIG. 11, to completely cover the cartilage-containing bone plug.

(2) In a modified procedure for the cartilage-containing bone plug preparation the femoral condyle was placed into a clamp, taking care not to damage the cartilage, the condyle was divided down the middle carefully using a bone saw. One hemi condyle was then returned to a glass beaker and work was continued with the other hemi condyle. Starting from the posterior end of the hemi condyle, a first osteochondral cartilage-containing bone plug was drilled at a 90° angle to the cartilage's surface using a 15 mm drill bit. Drilling was continued with the remaining cartilage-containing bone plugs (up to four plugs) from the hemi condyle. A bone saw was used to cut through the bone layer at a right (90°) angle to the drilling, in order to release the cartilage-containing bone plugs from the condyle. Each cartilage-containing bone plug was then placed in a separate labeled 50 ml centrifuge tube containing PBS solution (0.9% NaCl, Sigma St. Louis, USA) and 1% antibiotics (Penicillin/Streptomycin/Nystatin, Biological Industries, Beit Haemek, Israel). These steps were repeated for the remaining condyles. All of the 50 ml tubes were then placed into a 4° C. refrigerator for storage.

The purpose of these methods of cartilage-containing bone plug placement was to enable the removal of the cartilage-containing bone plug from the tube following initial thawing, and, to ensure that the seeding occurs in the freezing solution prior to the freezing moving on to the cartilage-containing bone plug.

### Freezing Protocol

Tubes containing the osteochondral cartilage-containing bone plugs prepared as described above were refrigerated until they reached 4° C. (for about 45 minutes). A prototype MTG device (IMT Interface Multigrad technology Ltd. Israel) similar to the device disclosed in U.S. Pat. No. 5,873,254 was used. The device was adapted for use with cartilage and with 16 mm diameter standard test tubes with a screw-carrying stopper, or with a double open-ended container as disclosed herein (16 or 18 mm diameter, an embodiment of which is illustrated in FIG. 11) and was set as follows: One gradient (0° C. to −6° C.) was imposed on a first 10 mm long block of the device and another gradient (−6° C. to −40° C.) was imposed on a second 225 mm long block of the device.

In two separate experiments, (the first conducted with the standard test tubes having the screw carrying stopper, the second with the double open-ended container similar to container 500) each was seeded immediately after removal from the above 4° C. incubation by dipping it in liquid nitrogen (LN) to a depth of 1 cm for 20 seconds. Up to 5 tubes/containers at a time were then placed in the device and moved along the thermal gradients at a velocity of 0.05 mm/sec resulting in a cooling rate of 0.45° C./minute (with the cartilage portions of the cartilage-containing bone plugs being the leading ends of the cartilage-containing bone plugs) and into the collection chamber of the device where they were further cooled with liquid nitrogen (LN) vapor to a temperature of approximately −100° C. When the temperature of the tubes/containers reached between −80° C. and −100° C. they were transferred to LN storage.

### Thawing Protocol

Tubes/containers containing the frozen osteochondral cartilage-containing bone plugs were removed from LN and maintained at Room Temperature (RT) for 140 seconds. The tubes were then dipped for 20-40 seconds in a water bath (at 50° C.) in such manner as to prevent the water in the bath from entering the tube. The Tubes were then unplugged and the cartilage-containing bone plugs were gently pulled out of the tube either with the string that was attached to the screw or in the case of container 500 by removing the two end plugs 530 and pushing the frozen contents out of the body 510 using tweezers of a suitable rod. Ice was then gently taken off the cartilage-containing bone plug using forceps. Once most of the visible ice layer was removed, the cartilage-containing bone plugs were immersed in 50° C. PBS (a container of PBS situated inside a water bath at 50° C.) for 20 seconds, after which the cartilage-containing bone plugs were transferred to new tubes with PBS solution at RT.

### Washing Protocol

(1) When working with standard tubes having the screw-containing stopper, washing was performed at room temperature (RT), where the cartilage was incubated for 5 minutes in each of the following solutions. First in a solution of 0.5M sucrose in F12, then in 0.25M sucrose in F12 and finally in 0.125M sucrose in F12. The washed cartilage-containing bone plugs were transferred to F12.
(2) When working with the double open ended container 500, washing was performed at room temperature (RT), where the cartilage was incubated for 5 minutes in each of the following solutions. First in a solution of 5% dextrose in 0.9% NaCl (×3 washing cycles) and then the washed cartilage-containing bone plugs were transferred to F12.

### Results of the Cartilage Cutting Procedure:

1. The Effect of Cutting Cartilage on Cell Viability During Cryopreservation

### Human and Sheep Cartilage

In the following, results were obtained with a cartilage having cuts or incisions performed with a cutting pattern as illustrated in FIG. 2A and freezing of the cartilage in a standard test tube having a screw-containing stopper. As a control, cartilage with no cuts were used.

In order to examine the effect of cutting the cartilage on the viability of chondrocyte cells, several methods of preparing of cutting were investigated. Every method was tested on human and on sheep osteochondral cartilage-containing bone plugs, using two different freezing solutions, with and without ethylene glycol. One difference between human and sheep articular cartilage being that sheep cartilage is normally about 1 mm thick while human cartilage is about 4 mm thick. Thus, viability of thawed cells only in the top 100-150 μm of the cartilage would amount to much lower overall viability in human cartilage than in sheep. As a control, cartilage-containing bone plugs without cuts were used. Viability was assayed after thawing in cuts spanning the whole depth of the cartilage portion of the cartilage-containing bone plugs, and viability was assayed using the live/dead ration assay.

All cartilage-containing bone plugs which were not incised, had poor overall viability. Cell survival was limited to cells in depth of up to 50 μm in human and 200 μm in sheep from the surface of the superficial layer, but in that layer the survival was up to 90%. The average overall survival rate (i.e. for the whole volume of the cartilage portion of the tissue) was 5-20%. When freezing without EG in the freezing solutions, survival was reduced to about half as much.

All cartilage-containing bone plugs which were incised, both human and sheep, had 90-100% viability of chondrocyte cells in the area surrounding the incisions. The differences between the different groups were in the maximal distance from the incision to viable cells in the surrounding area and it was dependent on the type of incision and the presence of EG. Without using EG in the freezing solution, during cutting and during the freezing process itself, results on average were lower (by about 20%) compared to using freezing solution containing EG (both during cutting and freezing). These satisfactory results have a benefit that the cryogenically preserved cartilage containing tissue contains essentially no cryoprotectant agents such as EG or Dimethyl Sulfoxide (DMSO) (less than 10% weight/volume, preferably less than 5% weight/volume) during the entire freezing and thawing processes. The effect of different incision types on the maximal distance from the incision to surviving cells surrounding the incision is shown in Table 1:

TABLE 1
maximal Distance of Viable cells from Cuts

| Type of cut | Maximal distance from cut |
|---|---|
| Cutting with scalpel blade | Up to 50 μm |
| Puncture with 400 μm needle | Up to 70 μm |
| Manual cutting with razor blade | Up to 200 μm |

In continuing studies, the effect of different cuts of the cartilage portion of the cartilage-containing tissue using razor blades was evaluated. One method was to combine several blades together, with predetermined and equal distance between the blades, for example the system depicted in FIG. 10C. When the distance between adjacent blades was 500 or 400 μm, viable cells were observed up to a depth of about 100 μm on average from the cuts. However, when the distance between blades was 300 μm, the survival was even higher, up to a depth of about 150 μm on average from the cuts. Accordingly, when the distance between the blades was about 300 μm, the whole area between the blades had 90-100% viability of the cells. The higher length may be due to some synergism between the cuts or higher pressure on the tissue during cutting.

Another observation was that although the razor blade width is 100 μm, the cut width was only 10-20 μm. This is probably because the cut was initiated by the sharp end (the cutting edge) of the blade, the width of which is much smaller than the width of the blade. When the remaining part of the blade enters the cartilage with its full width, it pushes the tissue aside.

However, it should be noted that other micro tools can be used to provide similar effect and such tools may have a sharp end which is below 10 μm, even lower as 1 μm. Another mean for producing such cuts can be by laser beam.

In yet a further assay, viability parameters of porcine-derived osteochondral cartilage-containing bone plugs using a cutting blade shown in FIG. 10B and a freezing protocol and washing protocol using the double-stopper tube as described above were determined.

Results showed chondrocyte viability of 53%±9% under regular fluorescent microscope, viable cell density of 18900±4100 cell/mm3, 68%±5.7% viability using a confocal microscope, which enables scanning of thicker samples, as compared to samples employed for regular microscope, thus reducing the damage caused to cells during the preparation of samples for microscopic imaging. This may explain the difference in viability obtained by the two imaging techniques employed herein. The results also showed ³⁵SO₄ uptake of 59% compared to fresh control. Biomechanical measures were mildly impaired (62%±5.2%) compared to fresh control due to the injection of cryoprotectants. In addition, chondrocyte viability in the cryopreserved allograft was preferentially maintained in the superficial zone. Similar results were obtained in human in-vitro studies.

In yet a further assay, viability parameters of human-derived osteochondral cartilage-containing bone plugs was determined. Eight 15 mm cylindrical grafts were examined for cell viability using fluorescent confocal microscopy. Biomechanical assessment was performed on a second set of 9 grafts to determine the matrix instantaneous modulus of elasticity.

Chondrocyte viability of 55.5%±13.9% (n=8), was observed as compared to 77% viability in fresh samples. This indicates an average cell survival rate of 71%±18% during the cryopreservation process.

Biomechanical measures were mildly impaired (n=9) 76.4%±12.09% compared to fresh control due to the injection of cryoprotectants. In addition, chondrocyte viability in the cryopreserved allograft was preferentially maintained in the superficial zone.

Thus, cryopreservation using the freezing methods, apparatuses and systems of the invention enabled the preservation of viable cells within the collagen matrix. These cells were embedded in the supporting hyaline cartilage matrix with good mechanical stability.

2. The Effect of Cutting Cartilage on Weight of Cartilage-Containing Tissue.

Sheep osteochondral cartilage-containing bone plugs were prepared as described above and about a millimeter or two of the bone was sawed off, resulting in cartilage-containing bone plugs having about 1 mm cartilage portions atop 1 mm bone portions. The cartilage-containing bone plugs were dried by gentle wiping with absorbent paper and then weighed. Each cartilage-containing bone plug was sliced exposed to air (i.e. not inside a solution) and subsequently water was seen to seep out from the sample (from the surface of the cartilage). The cartilage-containing bone plugs were weighed again and then were soaked in freezing solution (10% EG in PBS) or PBS (physiological buffered solution) for 15 min. The plugs were then removed from the solution, dried as described above and weighed again. The results (in grams) are summarized in Table 2. The change in weight is in percentage as compared with the weight of the same cartilage-containing bone plug before cutting.

TABLE 2
Weight of Cut Osteochondral Cartilage-containing bone plugs

| | | After cutting | | After Solution | |
|---|---|---|---|---|---|
| Solution | Before cutting | weight | change | weight | change |
| Freezing | 0.5084 | 0.4888 | −3.86% | 0.5036 | 3.03% |
| Solution | 0.4563 | 0.4365 | −4.34% | 0.4533 | 3.85% |
| | 0.4021 | 0.3788 | −5.79% | 0.3851 | 1.66% |
| PBS | 0.5974 | 0.5762 | −3.55% | 0.5761 | −0.02% |
| | 0.377 | 0.3594 | −4.67% | 0.3623 | 0.81% |
| | 0.3298 | 0.3136 | −4.91% | 0.315 | 0.45% |

As control, the cartilage-containing bone plugs were dried as described above and then incubated in freezing solution for 15 minutes without cutting. The results are summarized in Table 3. As can be seen, the weight gain of un-cut cartilage was significantly lower than cut cartilage.

TABLE 3
Weight of Uncut Ostechondral Cartilage-containing bone plugs

| | | |
|---|---|---|
| Cartilage- | | |
| containing bone | weight before | After immersion |

| plug No. | immersion | Weight | Change |
|---|---|---|---|
| 1 | 0.2338 | 0.2352 | 0.60% |
| 2 | 0.3563 | 0.3560 | −0.08% |

3. The Effect of Cutting Cartilage on the Glycosaminoglycans Contents of Cartilage-Containing Tissue.

Three sheep osteochondral cartilage-containing bone plugs were dried as described above, and divided to the groups. After drying cartilage-containing bone plugs from Groups 1 and 2 were cut in F12 solution. During the same time cartilage-containing bone plugs from group 3 were incubated in F12 without cutting, to serve as control. The solutions were then assayed for the presence of glycosaminoglycans (GAGs) using the dimethylene blue (DMMB) (Farndale assay) method which quantifies sulphated glycosaminoglycans (which hold water molecules), mainly Chondroitin Sulfate (CS). The binding generates a color reaction which is proportional to the GAGs' concentration. The optical density (OD) is measured using a spectrophotometer.

TABLE 3
Excision of GAGs

| Cartilage-containing | | |
|---|---|---|
| bone plug Group | OD | CS (micrograms/ml) |
| 1 | 0.0188 | 5.628743 |
| 2 | 0.0185 | 5.538922 |
| 3 | 0.0007 | 0.209581 |

As seen in Table 3, significantly more GAGs were observed in the solutions of the cut cartilage-containing bone plugs than in that of the uncut one, leading to the conclusion that the cutting may cause release of GAGs.

4. The Effect of Cutting Cartilage on the Protein Content of Cartilage-Containing Tissue

Sheep's cartilage-containing bone plugs were sliced in a protein-free PBS solution. Colorimetric total protein assay was preformed using bovine serum Albumin (BSA) (Pierce Ltd) as a standard and cartilage-containing bone plugs with no slicing as controls. In the solution of the cut cartilage a significantly higher amount of protein was observed than in the uncut or cartilage-containing bone plug free control (not shown).

5. Ice Crystal Formation

In order to examine the mechanism of ice crystallization inside the cuts sheep osteochondral cartilage-containing bone plugs were prepared as described above, and cut while immersed in freezing solution (10% EG in PBS). Group A cartilage-containing bone plugs were frozen, and thawed without application of biological glue. In groups B and C, after cutting biological glue (histoacryl manufactured by BRAUN aesculap, a tissue adhesive material used clinically for wound closure) was applied to the top of the cartilage portion, such that it partially penetrated the cuts and glues their upper ends together. Group B cartilage-containing bone plugs were maintained in a refrigerator for 24 hours at 4° C., and not frozen, whilst Group C cartilage-containing bone plugs were frozen and thawed. Viability was measured using the live/dead ratio assay, and the results are depicted in Table 4:

TABLE 4
Effect of Biological Glue

| Group | Viability |
|---|---|
| A | above 70% viability in cartilage all layers. |
| B | viability about 100% |
| C | Cells around cuts in areas that were sealed by glue were all dead, |
| | Cells in deeper layers where glue did not penetrate showed 70% |
| | viability. |

As seen in Table 4, apparently exposure to the glue had no measured adverse effect on chondrocyte viability. Nevertheless, freezing and thawing of cells adjacent to the glue cause cell death. Not wishing to be bound by theory, it is hypothesized that the glue disrupts the formation of dendritic ice (since ice in the glue is known to be planar). Once the ice passed the glue, it could resume dendritic shape and thus the cells could survive at a rate similar to that of unglued cut cartilage-containing bone plugs.

6. The Effect of Cutting Cartilage on Osteochondral Allograft Transplanted into the Knee of a Sheep

The purpose of this experiment was to evaluate functionality and viability of frozen thawed cartilage-containing tissue after being prepared with cuts, including the ability of the tissue to remain viable and to recover and even to produce new hyaline cartilage, after transplantation, in the areas where the cuts were made.

Osteochondral cartilage-containing bone plugs that were obtained from slaughterhouse sheep (to be used as allografts) were frozen and thawed as described above. The first 2 cartilage-containing bone plugs were only partially cut while immersed in 10% EG freezing solution, therefore in each cartilage-containing bone plug, one section of the cartilage portion (about 50%) was cut, and the other section remained uncut. The second 2 cartilage-containing bone plugs were fully cut while immersed in 10% EG freezing solution. The cartilage-containing bone plugs were stored for 8 weeks at LN until being thawed.

For transplantation of the cartilage, four skeletally mature Assaf sheep were operated under general anesthesia by an orthopeadic surgeon, sheep lying supine, preparation and draping of the right or left knee, including shaving of the wool.

Using a conventional lateral para-patellar approach, a longitudinal incision of the skin and subcutaneous tissue was performed. A lateral arthrotomy was performed by extension of the incision through the para-patellar fascia, thereby exposing the patello-femoral joint. The patella was then medially everted in order to facilitate full exposure of both femoral condyles. The exposure was further enhanced by maximal knee flexion. Meticulous preservation of the common tendon of origin of the peroneus tertius and extensor digitorum longus muscles was performed.

Transplantation was performed using a drill 13 mm outer diameter. Accordingly, a 9.5 mm osteochondral cartilage-containing bone plug was removed from the central weight-bearing portion of the medial femoral condyle. The removed cartilage-containing bone plugs were placed in gauze soaked with normal saline, for subsequent transplantation as an autograft into the lateral femoral condyle as control.

The base of the defect formed by removal of the cartilage-containing bone plug was further deepened as necessary, in order to match the length of the allograft to be transplanted; this correct sizing allowed a smooth congruent articular surface. After copious irrigation with normal saline, the defect was then filled, using a press-fit technique, with transplantation of the thawed cryopreserved allograft. Similar drilling with a 9 mm drill was performed over the central weight bearing area of the lateral femoral condyle, taking care not to injure the medially placed common tendon of the peroneus tertius and extensor digitorum longus muscles. The cartilage-containing bone plug removed from the medial femoral condyle was then similarly transplanted as an autograft, into the lateral femoral condyle. After irrigation with normal saline and confirmation of haemostasis, the patella was reduced and the knee was placed through a full range of passive flexion and extension; this confirms congruency and press-fit stability of the transplanted cartilage-containing bone plugs. The lateral para-patellar fascia was then sutured using an absorbable vicryl 2.0 suture; the subcutaneous tissue was similarly sutured with vicryl 2.0. Marcaine was injected into the knee joint for early post-operative analgesia. Staples were used for skin closure followed by a bandage which was stabilized by suture to the surrounding wool. The sheep were removed from the operating table and taken to the recovery area.

Follow-ups were performed using different methods:

1. Once a month the sheep were observed and their knee functions were scored during standing, walking and Running.

2. The first sheep was sacrificed 8 weeks after transplantation and second sheep 10 weeks after transplantation and the last 2 sheep were sacrificed 12 month after transplantation. Their knees were carefully evaluated using different methods such as computerized tomography (CT scan), viability staining using fluorescence staining and different histological staining.

All sheep scored 5 points (maximal score) on their functional score 1 month post surgery and maintained this score until being sacrificed.

CT Arthrogram with telebrix injection to the intra articular space: the bony part of the cartilage transplant was well incorporated into the surrounding bone. There was good continuation between the cartilage of the transplant and the original cartilage of the recipient sheep surrounding the transplant.

Live/Dead Assay showed areas of live cells around the articular surface and in the vicinity of the cuts deeper in the cartilage.

Oseotome biopsies were decalcified and stained with hematoxilin eosin (H&E), alcain blue or manson trichrome—in H&E there were areas of partially necrotic hyaline cartilage with reparative changes around lightly colored hyaline areas (these light areas were separated from each other with a distance of approximately 0.5 mm and were compatible with the cuts made during preparation). Reparative changes included large groups of chondrocytes inside the lacunas with enlarged cellular nucleus. Alcain blue staining showed the filling of the cuts with bluish colorization indicating the presence of high proteoglycan concentrations in the filling matrix. Subchondral bone showed enchondrosification (transformation from cartilage to bone) in the top layer and some fibrocytes with intertrabecular fibrosis. The deeper layers of the bone showed intertrabecular fibrosis and proliferation of osteoblasts compatible with reparative changes. There are no signs of acute inflammation.

With the 2 sheep sacrificed 12 month after transplantation some additional tests were performed including immunohistochemical staining for collagen type I and collagen type II which showed normal production levels which indicate that the cartilage maintains the properties of hyaline cartilage.

A full scoring of the last 2 sheep biopsies was conducted with the O'driscol cartilage scoring method [O'Driscoll S W, Keeley F W, Salter R B. The chondrogenic potential of free autogenous periosteal grafts for biological resurfacing of major full-thickness defects in joint surfaces under the influence if continuous passive motion. An experimental investigation in the rabbit. Journal of Bone and Joint Surgery-American. 68(7):1017-1035, 1986.]

Table 5 provides the different scoring with the two last sheep:

TABLE 5

| | Location: | |
|---|---|---|
| | 1^{st} sheep | 2^{nd} sheep |
| | (#3400) | (#3617) |
| | Left Medial | Left Medial |
| Nature of predominant tissue | | |
| Cellular morphology | | |
| Hyaline articular cartilage | 4 | 4 |
| Incompletely differentiated mesenchyme | — | — |
| Fibrous tissue or bone | — | — |
| Safranin-O staining of the matrix | | |
| Normal or nearly normal | 3 | 3 |
| Moderate | — | — |
| Slight | — | — |
| None | — | — |
| Structural characteristics | | |
| Surface regularity | | |
| Smooth and intact | — | — |
| Superficial horizontal lamination | — | 2 |
| Fissures 25-100% of the thickness | 1 | — |
| Severe disruption, including fibrillation | — | — |
| Structural integrity | | |
| Normal or nearly normal | 3 | — |
| Slight disruption, including cysts | — | 1 |
| Severe disruption | — | — |
| Thickness | | |
| 100% of normal adjacent cartilage | 2 | — |
| 50-100% of normal cartilage | — | — |
| 0-50% of normal cartilage | — | 0 |
| Bonding to the adjacent cartilage | | |
| Bonded at both ends | — | |
| Bonded at one end, or partially at both ends | 1 | 1 |
| Not bonded | — | |
| Freedom from cellular changes of | | |
| degeneration | | |
| Hypocellularity | | |
| Normal cellularity | — | 3 |
| Slight hypocellularity | 2 | — |
| Moderate hypocellularity | — | — |
| Severe hypocellularity | — | — |
| Chondrocyte clustering | | |
| No clusters | — | 2 |
| <25% of the cells | 1 | — |
| 25-100% of the cells | — | — |
| Freedom from cellular changes of | | |
| degeneration in adjacent cartilage | | |
| Normal cellularity, no clusters, normal | 3 | 3 |
| staining | | |
| Normal cellularity, mild clusters, moderate | — | — |
| staining | | |
| Mild or moderate hypocellularity, slight | — | — |
| staining | | |
| Severe hypocellularity, poor or no staining | — | — |
| Collagen Localization and Staining | | |
| Collagen I | Normal | Normal |
| Collagen II | Normal | Normal |
| Total Score: | 20 | 19 |

From reviewing the scoring results it may be concluded that a large amount of viable cells were maintained, thus maintaining normal matrix biochemical properties.

All the above results show the merits of the osteochondral grafts. The sheep showed good functional capabilities after surgery, CT arthrogram demonstrated good integration between the osteochondral allograft and the surrounding cartilage. Histology studies with H&E (Hematoxylin & Eosin) demonstrated viable cartilage cells throughout the entire thickness of the cartilage with filling of the cuts in the cartilage with eosin (red) staining matrix. Alcian blue staining (Clark, G, (ED.), staining Procedures, 3rd Edition, Williams & Wilkins, Baltimore, p. 166, c 1973) showed the filling of the cuts with bluish colorization indicating the presence of high proteoglycan concentrations in the filling matrix. The filling of the cuts was seen in the 2 sheep sacrificed in the early stages of the study in the sheep sacrificed 12 month after transplantation the histology shows viable and functioning cells near the cuts but there was no filling of these cuts\fissures. Thus, the cryoprotectant injection system was revised to reduce the amount of mechanical injury to the tissue.

As for the allograft that was sliced only in half of its surface—the sheep had a high functional score, however the H&E stain shows necrotic cartilage in the areas that were not cut and cartilage with viable cells throughout the entire thickness in the areas that were cut.

7. The Effect of Cutting Cartilage on Hemi-Condyle or Full Condyle Osteochondral Allograft

For this experiment a distinct freezing protocol was used as indicated below, using a static bi-directional freezing device as disclosed in U.S. Ser. No. 60/600,804 (filed Aug. 12, 2004), and PCT IL 2005/000876, which are incorporated herein in full. Fresh cadaver sheep legs were purchased from a slaughter house (Holon Slaughter house, Israel), and all manipulations of tissue samples were done in a sterile manner. Hemi condyles were produced (35×20×18 mm size) by cutting the condyles and then maintained in a buffered physiological solution containing 0.9% NaCl (Sigma, St. Louis, USA) and 3% antibiotics (Penicillin/Streptomycin/Nystatin, Biological Industries, Beit Haemek, Israel) for 24 hours.

The hemi-condyles were then completely immersed in sterile freezing solution (10% EG in PBS) for 45 minutes. Parts of the cartilage portion of the hemi condyle were cut using a cutting head shown in FIG. 10A and a blade headshown in FIG. 10B, which was brought down on the cartilage section of hemi condyle such that the blades entered the cartilage from directly above and cut down in parallel cuts to the level of the bone. Since the condyle cartilage surface is round, the cuts did not uniformly reach its whole surface and in part of the cartilage portion they were deeper than in others.

Each hemi condyle was then placed in a plastic bag filled with freezing solution and sealed. The bag was placed in a static freezing device, being a prototype static directional freezing device (IMT Interface Multigrad technology Ltd. Israel) manufactured according to U.S. Ser. No. 60/600,804 (filed Aug. 12, 2004), and PCT IL 2005/000876. The device has a variable distance between its two freezing blocks. The distance was set to 20 mm and then the bag was placed between the block in way that the cartilage portion of the hemi-condyle was in direct contact (through the bag) with one cooling block of the device, and the opposite bone end of the hemi-condyle was in direct contact (through the bag) with the opposite cooling block The freezing blocks were initially at temperature of 10° C. and it went down, in a controlled manner, to 0° C. in 30 minutes. Then the blocks were cooled to −15° C. (the cooling time took less than 30 seconds) and they were held at that temperature for 20 minutes. Then the blocks were cooled to −40° C. at a cooling rate of 0.4° C./minute. Then the blocks were cooled down to −80° C. within 2 minutes and that temperature was maintained for 10 minutes. After that the bag containing the tissue was transferred to LN storage.

A similar study was done with an MTG freezing machine using a large volume diameter tube.

### Results:

Regardless which of the above freezing protocols, the results were similar. Areas of the cartilage portion that were cut with the blade array showed 75%-95% viability (down to the portion in contact with bone, where the cuts traversed the depth of the cartilage portion), similar to the cartilage-containing bone plugs. Areas that were not cut showed reduced viability between 0-20%.

8. Correlation Between Cutting Surface Area and Viability of Cartilage Cells

A study was performed on porcine cartilage so as to determine the effect of the type of cut (full or partial cut), or no cut on the viability of cells after cryopreservation. Partial cutting was performed with variable distances between the blades to produce different levels of surface injuries (the surface exposed after cutting). For each group, the percentage of surface injury using a simple geometric calculation was determined. After cryopreservation and thawing, two measurements were performed to assess tissue quality.

To determine cell viability, staining of the cells using fluorescent nucleic stains (syto13, PI) was performed. The ratio between membrane intact cells (viable green staining cells) to total cells (red+green) was calculated. Matrix stiffness was determined using a confined compression system at 15% strain the modulus of elasticity was calculated from the slope of the linear part of the strain stress curve [J W Riley et al. Chondrocyte survival and material properties of hypothermically stored cartilage. Am J Sport Med 32:132-139, 2004].

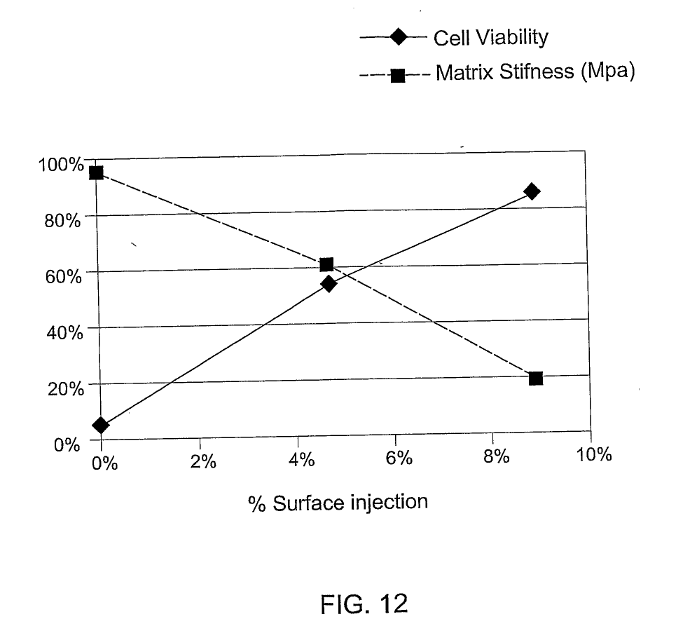

FIG. 12 provides the resulting curve, showing that when no incisions are performed there was minimal damage to the matrix stiffness (about 5% reduction) albeit, there was a layer of 200 micron of viable cells in the most superficial layer of the cartilage accounting for the 5% residual viability (most probably due to surface diffusion of the cryoprotectant). However, when full cuts were performed an estimate of 9% surface injury was obtained, which caused a severe reduction in matrix stiffness (80% reduction) albeit, a significant increase in cell viability, up to 90%. Intermediate injury levels (partial cuts) showed an inverse linear relation between the surface injury and matrix stiffness and a straight linear relation between surface injury and cell viability.

To summarize, when no cuts or incisions are performed a superficial layer of viable cells extending to a depth of 200 microns from the surface was observed. This was consistent with sheep cartilage and human cartilage. Thus, when considering cartilage thickness, the viability of sheep cartilage when no incisions were performed was about 20%, for porcine cartilage the viability would be about 10%, and for human cartilage the viability would be around 6%.

1.-37. (canceled)

38. A method for providing viable cartilage-containing tissue, comprising:
(a) providing excised cartilage-containing tissue; and
(b) treating said excised cartilage-containing and cryogenically preserving the treated cartilage-containing tissue under appropriate cryogenic preservation conditions so as to yield cryogenically preserved cartilage-containing tissue having at least 10% viable chondrocytes throughout the cartilage portion of the cartilage-containing tissue after preservation, as tested in a live/dead ratio assay.

39. A method for providing viable cartilage-containing tissue, comprising:
(a) providing excised cartilage-containing tissue having a cartilage portion;
(b) treating said excised cartilage-containing tissue by providing at least one incision in said cartilage portion to a predetermined depth therein; and
(c) cryogenically preserving the treated cartilage-containing tissue under appropriate cryogenic preservation conditions.

40. A method for providing viable cartilage-containing tissue, comprising:
(a) providing excised cartilage-containing tissue having a cartilage portion;
(b) treating said cartilage-containing tissue by introducing a cryoprotectant agent at least into said cartilage portion; and
(c) cryogenically preserving said treated cartilage-containing tissue under appropriate cryogenic preservation conditions.

41. The method of claim 38, wherein said treatment comprises providing a plurality of incisions in said cartilage portion to a predetermined depth therein.

42. The method of claim 40, wherein said treatment comprises providing a plurality of incisions in said cartilage portion to a predetermined depth therein.

43. The method of claim 39, wherein said predetermined depth comprises a depth of at least 50 μm from a surface of the cartilage portion.

44. The method of claim 42, wherein said predetermined depth comprises a depth of at least 200 μm from a surface of the cartilage portion.

45. The method of claim 43, wherein said predetermined depth does not exceed the local thickness of said cartilage portion.

46. The method of claim 43, wherein said at least one incision is formed by means of a cutting blade applied to the cartilage-containing tissue.

47. The method of claim 43, wherein said at least one incision is provided in an incision pattern over said cartilage portion comprising a plurality of individual incisions.

48. The method of claim 47, wherein said incision pattern comprises any one of the following patterns when viewed in a direction substantially perpendicular to said cartilage portion:
a plurality of substantially elongate channels in substantially parallel spaced relationship;
a plurality of substantially elongate channels radiating from a common central area;
a plurality of substantially concentric channels radiating from a common central area;
a plurality of mutually-spaced point incisions arranged in a suitable two dimensional matrix.

49. The method of claim 39, further comprising
(a) thawing the preserved cartilage containing tissue, the thawed cartilage containing tissue exhibiting at least 10% viable chondrocytes throughout the cartilage portion of the cartilage containing tissue.

50. The method of claim 39, wherein when said cartilage portion comprise a bone segment, the method comprises, prior to preservation:
providing a pulling member; and
connecting said pulling member to said bone portion.

51. The method of claim 39, comprising prior to cryogenic preservation introduction of the cartilage containing tissue into a vessel comprising:
a substantially impermeable body having a first open end and a second open end at longitudinally opposite ends thereof and defining a containing volume;
a first end plug and a second end plug for reversibly sealing said first open end and a second open end, respectively.

52. Cartilage-containing tissue, obtained with the method of claim 39.

53. The cartilage-containing tissue of claim 52, being thawed viable preserved human cartilage-containing tissue.

54. The cartilage-containing tissue of claim 53, being preserved for a period of at least 14 days.

55. A method of grafting a cartilage containing tissue, comprising providing a cartilage-containing tissue of claim 52, suitably thawing said cartilage-containing tissue, and grafting said thawed cartilage-containing tissue to an appropriate patient.

56. Apparatus for preparing a cartilage-containing tissue for subsequent cryogenic preservation, comprising
a holder for holding said cartilage-containing tissue;
a cutting head comprising at least one incision-forming element for forming an incision in a cartilage portion of said cartilage-containing tissue when held in said holder.

57. Vessel for containing a cartilage-containing tissue, comprising
a substantially impermeable body having a first open end and a second open end at longitudinally opposite ends thereof and defining a containing volume;
a first end plug and a second end plug for reversibly sealing said first open end and said second open end, respectively.
